SEQUENCE LISTING (37 sequences)

SEQ ID NO: 1 - DNA, 19 bp, Bacillus stearothermophilus
  misc_feature (1)..(6): N = G, A, C or T (U)
nnnnnngagtcnnnnnnnn

SEQ ID NO: 2 - DNA, 1815 bp, Bacillus stearothermophilus
  CDS (1)..(1812)
atggctaaaaaagttaattggtatgtttcttgttcacctagaagtccagaaaaaattcagcctgagttaaaagtactagcaaattttgagggaagttattggaaaggggtaaaagggtataaagcacaagaggcatttgctaaagaacttgctgctttaccacaattcttaggtactacttataaaaaagaagctgcattttctactcgagacagagtggcaccaatgaaaacttatggtttcgtatttgtagatgaagaaggttatcttcgtataactgaagcagggaaaatgcttgcaaataaccgaagacccaaagatgttttcttaaaacagttagtaaagtggcaatatccatcgtttcaacacaaaggtaaggaatatcccgaggaggaatggagtataaatcctcttgtatttgttcttagcttactaaaaaaggtaggcggcctcagtaaattagatattgctatgttctgtttaacagcaacaaataataatcaggtggatgaaattgcagaggaaataatgcagttccgtaatgaacgtgaaaaaataaaaggacaaaataagaaacttgagtttactgagaattacttttttaaaagattcgaaaagatttatggaaatgtaggtaaaattcgtgaagggaaatctgactcttcacataagtcaaaaattgaaactaaaatgagaaatgcacgagatgtggcagatgcaaccacaagatattttcgatatacaggtctatttgttgcaagagggaatcaactcgtcttaaatccagaaaaatctgatttaattgatgaaattatcagttcatcaaaagttgtaaagaactatacgagagtagaggaatttcatgaatattatggaaatccgagtttaccacagttttcatttgagacaaaagagcaacttttagatctagcccatagaatacgagatgaaaataccagactagctgagcaattagtagaacattttccaaatgttaaagttgaaatacaagtccttgaagacatttataattctcttaataaaaaagttgatgtagaaacattaaaagatgttatttaccatgctaaggaattacagctagaactcaaaaagaaaaagttacaagcagattttaatgacccacgtcaacttgaagaagtcattgaccttcttgaggtatatcatgagaaaaagaatgtgattgaagagaaaattaaagctcgcttcattgcaaataaaaatactgtatttgaatggcttacgtggaatggcttcattattcttggaaatgctttagaatataaaaacaacttcgttattgatgaagagttacaaccagttactcatgccgcaggtaaccagcctgatatggaaattatatatgaagactttattgttcttggtgaagtaacaacttctaagggagcaacccagtttaagatggaatcagaaccagtaacaaggcattatttaaacaagaaaaaagaattagaaaagcaaggagtagagaaagaactatattgtttattcattgcgccagaaatcaataagaatacttttgaggagtttatgaaatacaatattgttcaaaacacaagaattatccctctctcattaaaacagtttaacatgctcctaatggtacagaagaaattaattgaaaaaggaagaaggttatcttcttatgatattaagaatctgatggtctcattatatcgaacaactatagagtgtgaaagaaaatatactcaaattaaagctggtttagaagaaactttaaataattgggttgttgacaaggaggtaaggttttaa

SEQ ID NO: 3 - protein, 604 aa, Bacillus stearothermophilus
MAKKVNWYVSCSPRSPEKIQPELKVLANFEGSYWKGVKGYKAQEAFAKELAALPQFLGTTYKKEAAFSTRDRVAPMKTYGFVFVDEEGYLRITEAGKMLANNRRPKDVFLKQLVKWQYPSFQHKGKEYPEEEWSINPLVFVLSLLKKVGGLSKLDIAMFCLTATNNNQVDEIAEEIMQFRNEREKIKGQNKKLEFTENYFFKRFEKIYGNVGKIREGKSDSSHKSKIETKMRNARDVADATTRYFRYTGLFVARGNQLVLNPEKSDLIDEIISSSKVVKNYTRVEEFHEYYGNPSLPQFSFETKEQLLDLAHRIRDENTRLAEQLVEHFPNVKVEIQVLEDIYNSLNKKVDVETLKDVIYHAKELQLELKKKKLQADFNDPRQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 4 - DNA, 906 bp, Bacillus stearothermophilus
  CDS (1)..(903)
atgaaacctattttaaaatatcgtggtggaaaaaaagcagaaattcctttctttattgaccatatacccaatgatatcgaaacctactttgaaccctttgtcgggggtggtgctgtattcttccatttagaacatgaaaaatcagttatcaatgatattaattctaagctttataagttctatcttcaattaaagcacaattttgatgaggtaactaaacaattaaacgaactacaggaaatatatgaaaaaaaccaaaaggaatatgaggaaaaaaaagctcttgctcctgctggtgtcagagtggaaaataaaaatgaagaactatattatgagctaaggaacgaatttaactatccatcaggaaaatggctagacgcagtaatttattattttataaataaaactgcttatagtgggatgataaggtataacagtaaaggagaatataacgttccttttggaagatacaaaaactttaatacaaaaatcattactaaacaacaccataacctgcttcaaaaaacagaaatatataataaagatttttctgaaatttttaagatggcaaaaccaaatgacttcatgtttcttgatcctccatatgattgtatttttagtgattatggaaatatggagtttacaggtgatttcgacgagagggaacatcgtaggcttgctgaagagtttaaaaacttaaagtgccgtgcactaatgatcattagtaaaacggaattaactaccgaactatataaagattatatcgttgatgaatatcataaaagctattctgtaaacattagaaatagatttaagaatgaagcaaagcattatataatcaagaactatgattatgtacgaaaaaataaagaagaaaaatatgagcaacttgaacttattcattag

SEQ ID NO: 5 - protein, 301 aa, Bacillus stearothermophilus
MKPILKYRGGKKAEIPFFIDHIPNDIETYFEPFVGGGAVFFHLEHEKSVINDINSKLYKFYLQLKHNFDEVTKQLNELQEIYEKNQKEYEEKKALAPAGVRVENKNEELYYELRNEFNYPSGKWLDAVIYYFINKTAYSGMIRYNSKGEYNVPFGRYKNFNTKIITKQHHNLLQKTEIYNKDFSEIFKMAKPNDFMFLDPPYDCIFSDYGNMEFTGDFDEREHRRLAEEFKNLKCRALMIISKTELTTELYKDYIVDEYHKSYSVNIRNRFKNEAKHYIIKNYDYVRKNKEEKYEQLELIH

SEQ ID NO: 6 - DNA, 852 bp, Pseudomonas lemoignei
  CDS (1)..(849)
atgaagccattagttaaatatagaggtggaaagtctaaggaaattccatatctaattaaacatatccctgaatttaaagggcgctacatagagcctttttttggtgggggggctttatttttttatatagagccagaaaaatctattatcaatgacattaataaaaaacttatagatttttatcgagatgttaaagataactttgttcaattgcgtcatgagcttgatgagatagaatgtatttatgaaaagaatagagttgaatacgaaactagaaagaaattaaatcctactgaacgtgtagatgatggaaatgaagatttctattacttcatgaggaatgaattcaataaagatttttcggatagatatctttcatcaacactgtatttttatataaataagactgcgtactctggaatgattagatataactcaaaaggtgagtttaatgttccgtttggtagatataaaaatctcaatacaaaacttgtggctaatgaacatcacttgttaatgcagggtgctcagatatttaatgaagattacagcgagatcttcaagatggcgagaaaagatgattttatatttctagaccctccctatgattgcgtatttagtgattatggtaatgaggaatataaagatggtttcaatgtagatgctcatgtgaaattgagtgaggactttaagaaattgaaatgcaaagccatgatggttatcggtaagactgaattgactgatgggttgtataagaaaatgattattgatgaatacgataaaagttattctgtgaatataaggaatagatttaagtctgttgcaaagcatatagttgttgcaaattattga

SEQ ID NO: 7 - protein, 283 aa, Pseudomonas lemoignei
MKPLVKYRGGKSKEIPYLIKHIPEFKGRYIEPFFGGGALFFYIEPEKSIINDINKKLIDFYRDVKDNFVQLRHELDEIECIYEKNRVEYETRKKLNPTERVDDGNEDFYYFMRNEFNKDFSDRYLSSTLYFYINKTAYSGMIRYNSKGEFNVPFGRYKNLNTKLVANEHHLLMQGAQIFNEDYSEIFKMARKDDFIFLDPPYDCVFSDYGNEEYKDGFNVDAHVKLSEDFKKLKCKAMMVIGKTELTDGLYKKMIIDEYDKSYSVNIRNRFKSVAKHIVVANY

SEQ ID NO: 8 - DNA, 60 bp, Bacillus stearothermophilus
gtgaattcgagctcggtacccggggatcctctagagtcgacctgcaggcatgcaagcttg

SEQ ID NO: 9 - DNA, 59 bp, Bacillus stearothermophilus
ggtcgcggatccgaattcgagctccgtcgacaagcttgcggccgcactcgagcaccacc

SEQ ID NO: 10 - protein, 31 aa, Bacillus stearothermophilus
MAKKVNWYVSCSPWSPEKIQPELKVLANFEG

SEQ ID NO: 11 - protein, 12 aa, Bacillus stearothermophilus
  UNSURE (2): Xaa = any amino acid
MXIPYEDFADLG

SEQ ID NO: 12 - protein, 8 aa, Bacillus stearothermophilus
MAKKVNWY

SEQ ID NO: 13 - protein, 6 aa, Bacillus stearothermophilus
YEDFAD

SEQ ID NO: 14 - DNA, 22 bp, Bacillus stearothermophilus
  misc_feature (5): N = G, A, C or T(U)
tggcnaaraargtnaaytggta

SEQ ID NO: 15 - DNA, 17 bp, Bacillus stearothermophilus
  misc_feature (3): N = G, A, C or T(U)
tcngcraartcytcrta

SEQ ID NO: 16 - DNA, 24 bp, Bacillus stearothermophilus
ctcttcatcaataacgaagttgtt

SEQ ID NO: 17 - DNA, 25 bp, Bacillus stearothermophilus
ttacaaccagttactcatgccgcag

SEQ ID NO: 18 - DNA, 24 bp, Bacillus stearothermophilus
gagtgtgaaagaaaatatactcaa

SEQ ID NO: 19 - DNA, 27 bp, Bacillus stearothermophilus
tatagttgttcgatataatgagaccat

SEQ ID NO: 20 - DNA, 51 bp, Pseudomonas lemoignei
aaaactgcagataaggaggtgatcgtatgaagccattagttaaatatagag

SEQ ID NO: 21 - DNA, 33 bp, Pseudomonas lemoignei
cgcggatcctcaataatttgcaacaactatatg

SEQ ID NO: 22 - DNA, 48 bp, Bacillus stearothermophilus
cgcggatcctaaggaggtgatctaatggctaaaaaagttaattggtat

SEQ ID NO: 23 - DNA, 33 bp, Bacillus stearothermophilus
cccaagcttttaaaaccttacctccttgtcaac

SEQ ID NO: 24 - DNA, 36 bp, Escherichia coli
accgcatcgaatgcgagtcgaggacgacggccagtg

SEQ ID NO: 25 - DNA, 38 bp, Escherichia coli
cgattccgcaatgcgagtcgaggccatgattacgccaa

SEQ ID NO: 26 - DNA, 13 bp, Escherichia coli
cagtcacgacgtt

SEQ ID NO: 27 - DNA, 13 bp, Escherichia coli
cacaggaaacagc

SEQ ID NO: 28 - DNA, 37 bp, Unknown
  Description of Unknown Organismthe last 13 bases are from pUC19, the preceeding bases are random.
accgcatcgaatgcgagtcatgttacgacggccagtg

SEQ ID NO: 29 - DNA, 39 bp, Unknown
  Description of Unknown Organismthe last 15 bases are from pUC19, the preceeding bases are random.
cgattccgctccaggagtcactttccatgattacgccaa

SEQ ID NO: 30 - DNA, 37 bp, Unknown
  Description of Unknown Organismthe last 13 bases are from pUC19, the preceeding bases  are random.
accgcatcgaatgcggatcatgttacgacggccagtg

SEQ ID NO: 31 - DNA, 39 bp, Unknown
  Description of Unknown Organismthe last 15 bases are from pUC19, the preceeding bases are random.
cgattccgctccagggatcactttccatgattacgccaa

SEQ ID NO: 32 - DNA, 42 bp, Unknown
  Description of Unknown Organismthe last 13 bases are from pUC19, the preceeding bases  are random.
accgcatcgaatatgtatcgccctcagctacgacggccagtg

SEQ ID NO: 33 - DNA, 44 bp, Unknown
  Description of Unknown Organismthe last 15 bases are from pUC19, the preceeding bases are random.
cgattccgctccagacttatccctcagctccatgattacgccaa

SEQ ID NO: 34 - DNA, 42 bp, Unknown
  Description of Unknown Organismthe last 13 bases are from pUC19, the preceeding are random
accgcatcgaatatgtatcgcgctgaggtacgacggccagtg

SEQ ID NO: 35 - DNA, 44 bp, Unknown
  Description of Unknown Organismthe last 15 bases are from pUC19, the preceeding bases are random.
cgattccgctccagacttatcgctgaggtccatgattacgccaa

SEQ ID NO: 36 - DNA, 38 bp, Unknown
  Description of Unknown Organismthe last 13 bases are from pUC19, the preceeding bases are random
accgcatcgaatgcatgtaccggctacgacggccagtg

SEQ ID NO: 37 - DNA, 40 bp, Unknown
  Description of Unknown Organismthe last 15 bases are from pUC19, the preceeding bases are random.
cgattccgctccagacttaccggctccatgattacgccaa

(19) United States
(12) Patent Application Publication
(10) Pub. No.: US 2003/0211506 A1
(43) Pub. Date: Nov. 13, 2003

(54) N. bstnbi nicking endonuclease and methods for using endonucleases in single-stranded displacement amplification

(76) Inventors: Huimin Kong, Wenham, MA (US); Lauren Sears Higgins, Rockport, MA (US); Michael A Dalton, Manchester, MA (US); Rebecca B Kucera, Hamilton, MA (US); Ira Schildkraut, Cerrillos, NM (US); Geoffrey G Wilson, Boxford, MA (US)

Correspondence Address: NEW ENGLAND BIOLABS, INC., 32 TOZER ROAD, BEVERLY, MA, 01915, US

(21) Appl. No.: 10/276,289

(22) PCT Filed: Jun. 1, 2001
(86) PCT No.: PCT/US01/17804

(51) Int. Cl. C12Q 1/68; C07H 21/04; C12P 19/34; C12N 9/22; C12N 1/21; C12P 21/02; C12N 15/74

(52) U.S. Cl. 435/6; 435/91.2; 536/23.2; 435/320.1; 435/252.3; 435/69.1; 435/199

(57) ABSTRACT

The present invention relates to recombinant DNA which encodes a novel nicking endonuclease, N.BstNBI, and the production of N.BstNBI restriction endonuclease from the recombinant DNA utilizing PleI modification methylase. Related expression vectors, as well as the application of N.BstNBI and other nicking enzymes in non-modified strand displacement amplification, is disclosed also.

### BACKGROUND OF THE INVENTION

The present invention relates to the recombinant DNA which encodes the N.BstNBI nicking endonuclease and modification methylase, and the production of N.BstNBI nicking endonuclease from the recombinant DNA. N.BstNBI nicking endonuclease is originally isolated from Bacillus stearothermophilus. It recognizes a simple asymmetric sequence, 5′ GAGTC 3′, and it cleaves only one DNA strand, 4 bases away from the 3′-end of its recognition site.

The present invention also relates to the use of nicking endonucleases in strand-displacement amplification application (SDA). More particularly, it relates to liberating such amplification from the technical limitation of employing modified (particularly α-thiophosphate substituted) nucleotides.

Restriction endonucleases are enzymes that recognize and cleave specific DNA sequences. Usually there is a corresponding DNA methyltransferase that methylates and therefore protects the endogenous host DNA from the digestion of a certain restriction endonuclease. Restriction endonucleases can be classified into three groups: type I, II, and III. More than 3000 restriction endonucleases with over two hundred different specificities have been isolated from bacteria (Roberts and Macelis, Nucleic Acids Res. 26:338-350 (1998)). Type II and type IIs restriction enzymes cleave DNA at a specific position, and therefore are useful in genetic engineering and molecular cloning.

Most restriction endonucleases catalyze double-stranded cleavage of DNA substrates via hydrolysis of two phosphodiester bonds on two DNA strands (Heitman, Genetic Engineering 15:57-107 (1993)). For example, type II enzymes, such as EcoRI and EcoRV, recognize palindromic sequences and cleave both strands symmetrically within the recognition sequence. Type IIs endonucleases recognize asymmetric DNA sequences and cleave both DNA strands outside of the recognition sequence.

There are some proteins in the literature which break only one DNA strand and therefore introduce a nick into the DNA molecule. Most of those proteins are involved in DNA replication, DNA repair, and other DNA-related metabolisms (Kornberg and Baker, DNA replication. 2nd edit. W. H. Freeman and Company, New York, (1992)). For example, gpII protein of bacteriophage fI recognizes and binds a very complicated sequence at the replication origin. It introduces a nick in the plus strand, which initiates rolling circle replication, and it is also involved in circularizing the plus strand to generate single-stranded circular phage DNA. (Geider et al., J. Biol. Chem. 257:6488-6493 (1982); Higashitani et al., J. Mol. Biol. 237:388-400 (1994)). Another example is the MutH protein, which is involved in DNA mismatch repair in E. coli. MutH binds at dam methylation sites (GATC), where it forms a protein complex with nearby MutS which binds to a mismatch. The MutL protein facilitates this interaction and this triggers single-stranded cleavage by MutH at the 5′ end of the unmethylated GATC site. The nick is then translated by an exonuclease to remove the mismatched nucleotide (Modrich, J. Biol. Chem. 264:6597-6600 (1989)).

The nicking enzymes mentioned above are not very useful in the laboratory for manipulating DNA due to the fact that they usually recognize long, complicated sequences and usually associate with other proteins to form protein complexes which are difficult to manufacture. Thus none of these nicking proteins are commercially available. Recently, we have found a nicking protein, N.BstNBI, from the thermophilic bacterium Bacillus stearothermophilus, which is an isoschizomer of N.BstSEI (Abdurashitov et al., Mol. Biol. (Mosk) 30:1261-1267 (1996)). Unlike gpII and MutH, N.BstNBI behaves like a restriction endonuclease. It recognizes a simple asymmetric sequence, 5′ GAGTC 3′, and it cleaves only one DNA strand, 4 bases away from the 3′-end of its recognition site (FIG. 1A).

Because N.BstNBI acts more like a restriction endonuclease, it should be useful in DNA engineering. For example, it can be used to generate a DNA substrate containing a nick at a specific position. N.BstNBI can also be used to generate DNA with gaps, long overhangs, or other structures. DNA templates containing a nick or gap are useful substrates for researchers in studying DNA replication, DNA repair and other DNA related subjects (Kornberg and Baker, DNA replication. 2nd edit. W. H. Freeman and Company, New York, (1992)). A potential application of the nicking endonuclease is its use in strand displacement amplification (SDA), which is an isothermal DNA amplification technology. SDA provides an alternative to polymerase chain reaction (PCR), and it can reach 10⁶-fold amplification in 30 minutes without thermo-cycling (Walker et al., Proc. Natl. Acad. Sci. USA 89:392-396 (1992)). SDA uses a restriction enzyme to nick the DNA and a DNA polymerase to extend the 3′-OH end of the nick and displace the downstream DNA strand (Walker et al., (1992)). The SDA assay provides a simple (no temperature cycling, only incubation at 60° C.) and very rapid (as short as 15 minutes) detection method and can be used to detect viral or bacterial DNA. SDA is being introduced as a diagnostic method to detect infectious agents, such as Mycobacterium tuberculosis and Chlamydia trachomatis (Walker and Linn, Clin. Chem. 42:1604-1608 (1996); Spears et al., Anal. Biochem. 247:130-137 (1997)).

For SDA to work, a nick has to be introduced into the DNA template by a restriction enzyme. Most restriction endonucleases make double-stranded cleavages. Therefore, modified α-thio deoxynucleotides (dNTPαS) have to be incorporated into the DNA, so that the endonuclease only cleaves the unmodified strand which is within the primer region (Walker et al., 1992). The α-thio deoxynucleotides are eight times more expensive than regular dNTPs (Pharmacia), and are not incorporated well by the Bst DNA polymerase as compared to regular deoxynucleotides (J. Aliotta, L. Higgins, and H. Kong, unpublished observation).

Alternatively, in accordance with the present invention, it has been found that if a nicking endonuclease is used in SDA, it will introduce a nick into the DNA template naturally. Thus the dNTPαS is no longer needed for the SDA reaction when a nicking endonuclease is being used. This idea has been tested, and the result agreed with our speculation. The target DNA can, for example, be amplified in the presence of the nicking endonuclease N.BstNBI, dNTPs, and Bst DNA polymerase. Other nicking endonucleases can also be used. It is even possible to employ a restriction endonuclease in which the two strands are cleaved sequentially, such that nicked intermediates accumulate.

With the advent of genetic engineering technology, it is now possible to clone genes and to produce the proteins that they encode in greater quantities than are obtainable by conventional purification techniques. Type II restriction-modification systems are being cloned with increasing frequency. The first cloned systems used bacteriophage infection as a means of identifying or selecting restriction endonuclease clones (EcoRII: Kosykh et al., Molec. Gen. Genet 178:717-719 (1980); HhaII: Mann et al., Gene 3:97-112 (1978); PstI: Walder et al., Proc. Nat. Acad. Sci. 78:1503-1507 (1981)). Since the presence of restriction-modification systems in bacteria enable them to resist infection by bacteriophages, cells that carry cloned restriction-modification genes can, in principle, be selectively isolated as survivors from libraries that have been exposed to phage. This method has been found, however, to have only limited value. Specifically, it has been found that cloned restriction-modification genes do not always manifest sufficient phage resistance to confer selective survival.

Another cloning approach involves transferring systems initially characterized as plasmid-borne into E. coli cloning plasmids (EcoRV: Bougueleret et al., Nucl. Acids Res. 12:3659-3676 (1984); PaeR7: Gingeras and Brooks, Proc. Natl. Acad. Sci. USA 80:402-406 (1983); Theriault and Roy, Gene 19:355-359 (1982); PvuII: Blumenthal et al., J. Bacteriol. 164:501-509 (1985)).

A further approach which is being used to clone a growing number of systems involves selection for an active methylase gene (refer to U.S. Pat. No. 5,200,333 and BsuRI: Kiss et al., Nucl. Acids Res. 13:6403-6421 (1985)). Since restriction and modification genes are often closely linked, both genes can often be cloned simultaneously. This selection does not always yield a complete restriction system however, but instead yields only the methylase gene (BspRI: Szomolanyi et al., Gene 10:219-225 (1980); BcnI: Janulaitis et al, Gene 20:197-204 (1982); BsuRI: Kiss and Baldauf, Gene 21:111-119 (1983); and MspI: Walder et al., J. Biol. Chem. 258:1235-1241 (1983)).

Another method for cloning methylase and endonuclease genes is based on a colorimetric assay for DNA damage (see U.S. Pat. No. 5,492,823). When screening for a methylase, the plasmid library is transformed into the host E. coli strain such as AP1-200. The expression of a methylase will induce the SOS response in an E. coli strain which is McrA+, McrBC+, or Mrr+. The AP1-200 strain is temperature sensitive for the Mcr and Mrr systems and includes a lac-Z gene fused to the damage inducible locus of E. coli. The detection of recombinant plasmids encoding a methylase or endonuclease gene is based on induction at the restrictive temperature of the lacz gene. Transformants encoding methylase genes are detected on LB agar plates containing X-gal as blue colonies. (Piekarowicz et al., Nucleic Acids Res. 19:1831-1835 (1991) and Piekarowicz et al., J. Bacteriology 173:150-155 (1991)). Likewise, the E. coli strain ER1992 contains a dinD1-LacZ fusion but is lacking the methylation dependent restriction systems McrA, McrBC and Mrr. In this system (called the “endo-blue” method), the endonuclease gene can be detected in the absence of its cognate methylase when the endonuclease damages the host cell DNA, inducing the SOS response. The SOS-induced cells form deep blue colonies on LB agar plates supplemented with X-gal. (Fomenkov et al., Nucleic Acids Res. 22:2399-2403 (1994)).

Sometimes the straight-forward methylase selection method fails to yield a methylase (and/or endonuclease) clone due to various obstacles (see, e.g., Lunnen et al., Gene 74(1):25-32 (1988)). One potential obstacle to cloning restriction-modification genes lies in trying to introduce the endonuclease gene into a host not already protected by modification. If the methylase gene and endonuclease gene are introduced together as a single clone, the methylase must protectively modify the host DNA before the endonuclease has the opportunity to cleave it. On occasion, therefore, it might only be possible to clone the genes sequentially, methylase first then endonuclease (see U.S. Pat. No. 5,320,957).

Another obstacle to cloning restriction-modification systems lies in the discovery that some strains of E. coli react adversely to cytosine or adenine modification; they possess systems that destroy DNA containing methylated cytosine (Raleigh and Wilson, Proc. Natl. Acad. Sci. USA 83:9070-9074 (1986)) or methylated adenine (Heitman and Model, J. Bacteriology 196:3243-3250 (1987); Raleigh et al., Genetics 122:279-296 (1989); Waite-Rees et al., J. Bacteriology 173:5207-5219 (1991)). Cytosine-specific or adenine-specific methylase genes cannot be cloned easily into these, strains, either on their own, or together with their corresponding endonuclease genes. To avoid this problem it is necessary to use mutant strains of E. coli (McrA− and McrB− and Mrr−) in which these systems are defective.

An additional potential difficulty is that some restriction endonuclease and methylase genes may not express in E. coli due to differences in the transcription machinery of the source organism and E. coli, such as differences in promoter and ribosome binding sites. The methylase selection technique requires that the methylase express well enough in E. coli to fully protect at least some of the plasmids carrying the gene.

Because purified restriction endonucleases, and to a lesser extent modification methylases, are useful tools for characterizing genes in the laboratory, there is a commercial incentive to obtain bacterial strains through recombinant DNA techniques that synthesize these enzymes in abundance. Such strains would be useful because they would simplify the task of purification as well as provide the means for production in commercially useful amounts.

### SUMMARY OF THE INVENTION

A unique combination of methods was used to directly clone the N.BstNBI endonuclease gene and express the gene in an E. coli strain premodified by PleI methylase. To clone the N.BstNBI endonuclease gene directly, both the N-terminal amino acid sequence and a stretch of internal amino acid sequence of highly purified native N.BstNBI restriction endonuclease were determined. Degenerate primers were designed based on the amino acid sequences, and PCR techniques were used to amplify a segment of the DNA gene that encodes the N.BstNBI endonuclease protein. The PCR product was sequenced, and the information was used to design primers for inverse PCR reactions. By chromosome walking via inverse PCR, the endonuclease open reading frame, n.bstNBIR, was deduced. Continuing with inverse PCR, an open reading frame was found adjacent to the endonuclease gene. Blast analysis suggested that this gene encoded an adenine methylase (n.bstNBIM).

The N.BstNBI endonuclease gene was cloned into a low copy-number T7 expression vector, pHKT7, and transformed into an E. coli host which had been premodified by a pHKUV5-PleI methylase clone. This recombinant E. coli strain (NEB#1239) produces about 4×10⁷ units N.BstNBI endonuclease per gram cell.

The present invention also relates to a novel method of DNA amplification. The method of using nicking endonuclease such as N.BstNBI in the absence of modified nucleotides such as α-thio dNTPs in strand displacement amplification is disclosed.

Additional examples of non-modified strand displacement amplification mediated by four additional enzymes generated by engineering of other nucleases is also disclosed. An example of non-modified strand displacement amplification mediated by a restriction endonuclease with a nicked intermediate is disclosed. Finally, approaches for constructing such nicking endonucleases are disclosed.

### BRIEF DESCRIPTION OF THE DRAWINGS

FIG. 1A shows the recognition sequence (SEQ ID NO: 1) and site of cleavage of N.BstNBI nicking endonuclease. N.BstNBI recognizes a simple asymmetric sequence, 5′ GAGTC 3′, and it cleaves only one DNA strand, 4 bases away from the 3′-end of its recognition site, indicated by the arrow head.

FIG. 1B shows the gene organization of N.BstNBI restriction-modification system where n.bstNBIR (R) is the N.BstNBI restriction endonuclease gene and n.bstNBIM (M) is the N.BstNBI modification methyltransferase gene.

FIG. 2 shows the DNA sequence of n.bstNBIR gene and its encoded amino acid sequence (SEQ ID NO: 2 AND SEQ ID NO: 3).

FIG. 3 shows the DNA sequence of n.bstNBIM gene and its encoded amino acid sequence (SEQ ID NO: 4 and SEQ ID NO: 5).

FIG. 4 shows the DNA sequence of pleIM gene and its encoded amino acid sequence (SEQ ID NO: 6 and SEQ ID NO: 7).

FIG. 5 shows the cloning vectors of pHKUV5 (SEQ ID NO: 8).

FIG. 6 shows the cloning vectors of pHKT7 (SEQ ID NO: 9).

FIG. 7 shows the result of non-modified strand displacement amplification using nicking enzyme N.BstNBI. Lane 1 shows the molecular weight standards and Lane 2 shows the 160-bp DNA fragment produced from SDA by N.BstNBI, which is indicated by the arrow head.

FIG. 8 shows the result of non-modified strand displacement amplification using five nicking enzymes, with duplicate samples run. Lanes 1 and 12 are the molecular weight marker lanes (100 bp ladder). Lanes 2 and 3, N.BstNBI; lanes 4 and 5, N.AlwI; lanes 6 and 7 N.MlyI; lanes 8 and 9, N.BbvCI-1-35; lanes 10 and 11, BbvCI-2-12. Arrow indicates the position of the expected 100-120 bp product bands.

FIG. 9 shows the result of non-modified strand displacement amplification using BsrFI, an enzyme that cleaves in two steps. Panel A, SDA reactions as described in Example 6 with: lane 1, no DNA substrate, no product appearing; lane 2, no BsrFI, no product appearing; lane 3, complete reaction, 150 bp amplicon appearing. M=size standard markers HaeIII digest of φX174; Panel B, SDA reactions as described in Example 6 but with different DNA substrates leading to different sized amplicons: Lane 1, 150 bp product; lane 2-190 bp product; lane 3-330 bp product; lane 4-430 bp product; lane 5-500 bp product. M=size standard markers HaeIII digest of φX174.

### DETAILED DESCRIPTION OF THE INVENTION

In accordance with one embodiment of this invention, procedures to identify and create site-specific nicking enzymes are described, and suitability of their application to SDA in the absence of modified nucleotides such as α-thio nucleotides is demonstrated.

Those skilled in the art will appreciate that for use in SDA, a nicking enzyme must have sequence-specificity in that activity, so that a single nick can be introduced at the location of the desired priming site. In SDA as conventionally applied, the sequence-specific nicking activity derives from two factors: the sequence-specificity of the restriction endonuclease employed and the strand-specificity enforced by the employment of modified (e.g. 60 -thiophosphate substituted, boron-substituted (α-boronated) dNTPs or cytosine-5 dNTP) nucleotides. This procedure increases the cost (due to the expense of the modified nucleotides) and reduces the length of the amplicon that can be synthesized (due to poor incorporation by the polymerase).

In the present invention, it is demonstrated that appropriate cleavage specificity can be enabled in other general ways. Five examples of such enzymes are disclosed in the present invention, obtained in four different ways.

In one preferred embodiment, both sequence specificity and strand specificity are obtained in an enzyme as found in the original host, exemplified by N.BstNBI.

The cloning of the N.BstNBI restriction endonuclease gene from Bacillus stearothermophilus 33M (NEB #928, New England Biolabs, Inc., Beverly, Mass.) proved to be challenging. A methylase selection strategy was tried and one methylase expression clone was isolated. However, the flanking ORFs did not encode the N.BstNBI nicking enzyme. This turned out to be an orphan methylase, i.e., a methylase not associated with the cognate endonuclease gene. The method by which the N.BstNBI nicking endonuclease was preferably cloned and expressed in E. coli is described herein:

1. Purification of the N.BstNBI restriction endonuclease to near homogeneity and N-terminal and internal amino acid sequence determination.

Nine chromatography columns were used to purify the N.BstNBI endonuclease protein. They included an XK 50/14 fast flow P-cell column, an HR 16/10 Source™ 15Q, five HR 16/10 Heparin-TSK-Guardgel columns, an HR 10/10 Source™ 15Q column and a Resource™ 15S. The purification yielded one protein band at approximately 72 kDa on an SDS-PAGE protein gel following Coomassie blue staining. The N-terminal 31 amino acid residues were determined by sequential degradation of the purified protein on an automated sequencer. To determine its internal protein sequence, a 6-kDa polypeptide fragment was obtained following cyanogen bromide digestion of the 72-kDa N.BstNBI protein. The first 13 amino acid residues of this 6-kDa were determined. This 13-amino acid sequence differs from the sequence of the N-terminal 31 amino acid residues, suggesting it was internal N.BstNBI protein sequence.

2. Amplification of a segment of the N.BstNBI endonuclease gene and subsequent cloning.

Degenerate primers were designed based on both the N-terminal and internal amino acid sequences. These primers were used to PCR amplify the 5′ end of the endonuclease gene. PCR products were cloned into plasmid pCAB16 and sequenced. The approximately 1.4 kb PCR fragment was then identified by comparing the amino acid sequences deduced from the cloned DNA with the N-terminal amino acid sequence of the N.BstNBI endonuclease protein.

3. Chromosome walking via inverse PCR to isolate the N.BstNBI endonuclease and methylase gene.

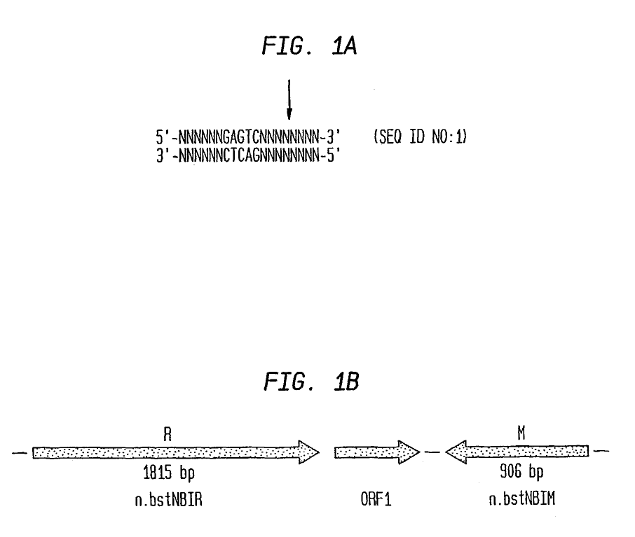

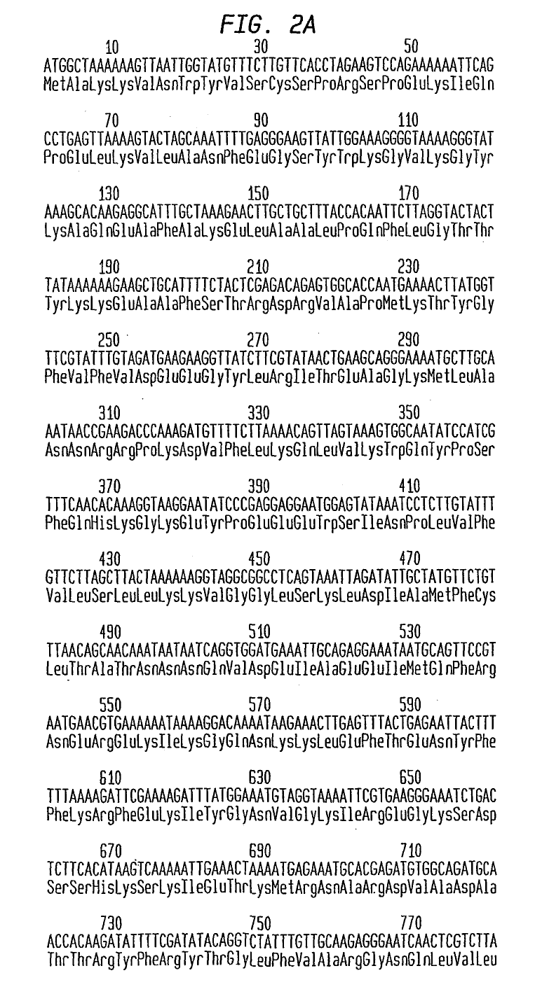

To clone the entire N.BstNBI endonuclease gene as well as its corresponding DNA methylase gene, inverse PCR techniques were adopted to amplify DNA adjacent to the original 1.4 kb endonuclease gene fragment (Ochman et al., Genetics 120:621 (1988); Triglia et al., Nucl. Acids Res. 16:8186 (1988) and Silver and Keerikatte, J. Cell. Biochem. (Suppl.) 13E:306, Abstract No. WH239 (1989)). In total, two rounds of inverse PCR were performed. At that point, the endonuclease and the methylase open reading frames (ORF) were identified (FIG. 1B).

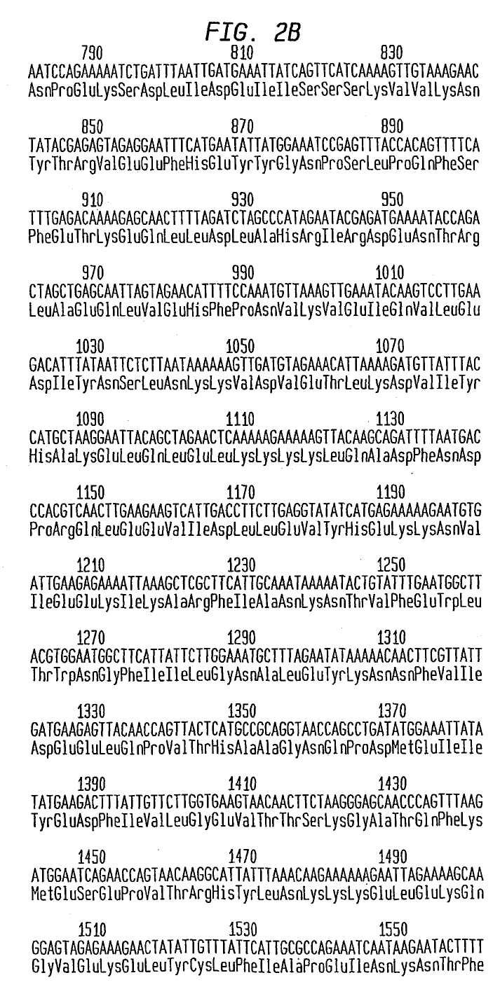

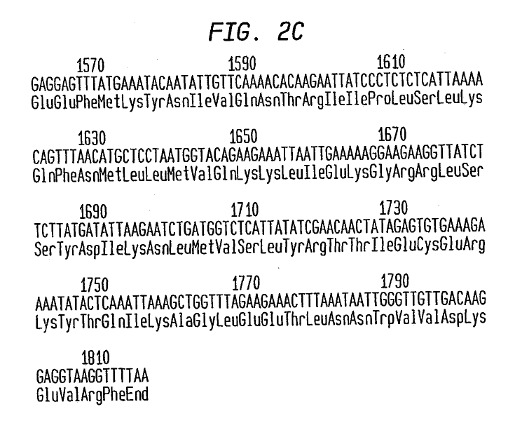

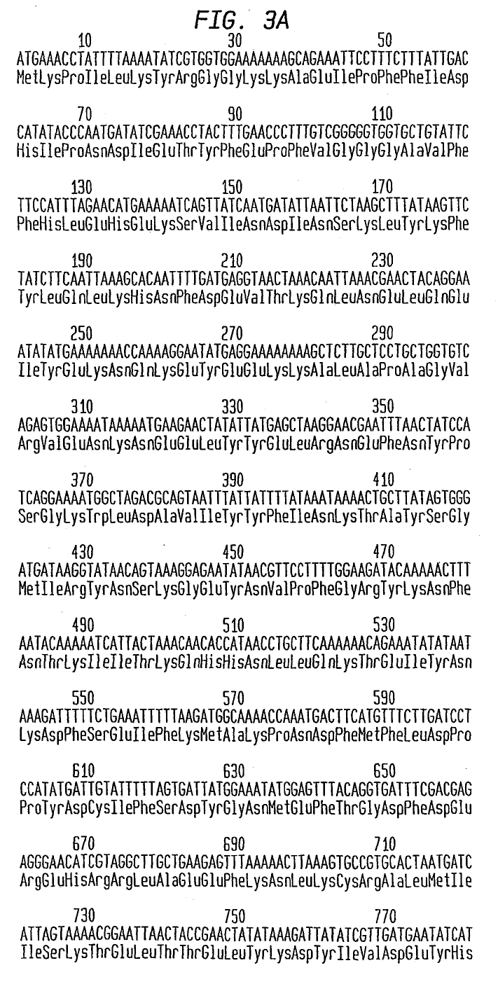

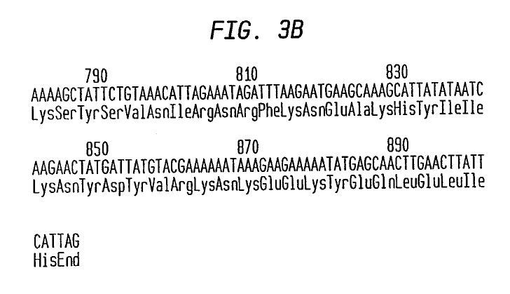

The endonuclease gene (n.bstNBIR) turned out to be a 1815-bp ORF that codes for a 604-amino acid protein with a deduced molecular weight of 70,368 Daltons (FIG. 2). This agreed with the observed molecular mass of the N.BstNBI endonuclease that was purified from native Bacillus Stearothermophilus 33M. Close to the endonuclease gene a 906-bp ORF, n.bstNBIM, was found. It was oriented in a convergent manner relative to the endonuclease (FIG. 1B). The protein sequence deduced from the n.bstNBIM gene shares significant sequence similarity with other adenine methylases (FIG. 3).

4. Expression of N.BstNBI endonuclease gene using pHKUV5 and pHKT7 plasmids.

The two-step method for cloning restriction-modification systems is described in U.S. Pat. No. 5,320,957. The first step is protection of the host cell from corresponding endonuclease digestion by pre-modification of recognition sequences. This is accomplished by introducing the methylase gene into a host cell and expressing the gene therein. The second step includes introduction of the endonuclease gene into the pre-modified host cell and subsequent endonuclease production.

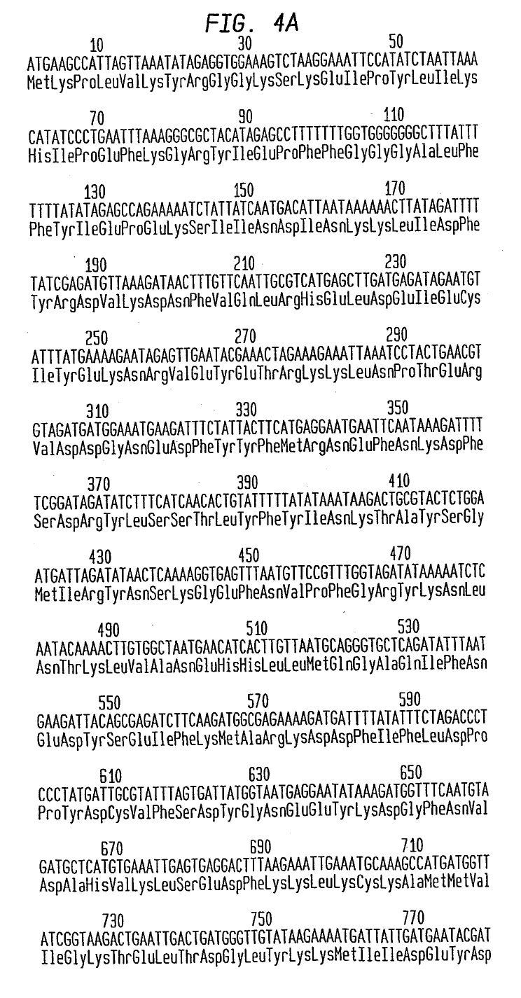

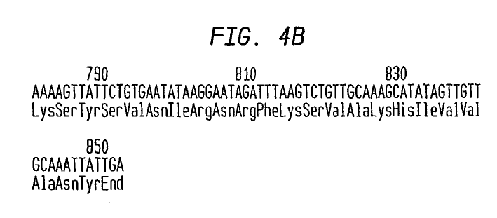

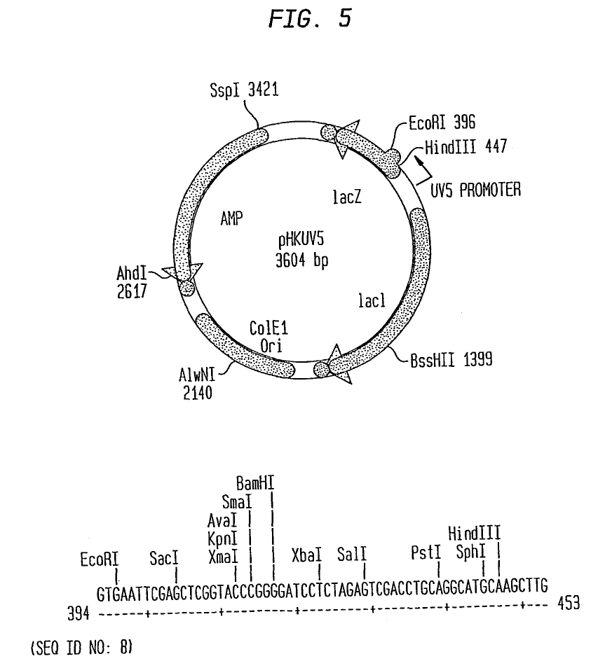

The pleIM gene (FIG. 4) was cloned into plasmid pHKUV5 (FIG. 5) and transformed into E. coli cells. As a result, the E. coli cells were modified by the pHKUV5-pleIM. In this case, the PleI methylase (pleIM) was used for pre-modification of the host cells because PleI and N.BstNBI share the same recognition sequence.

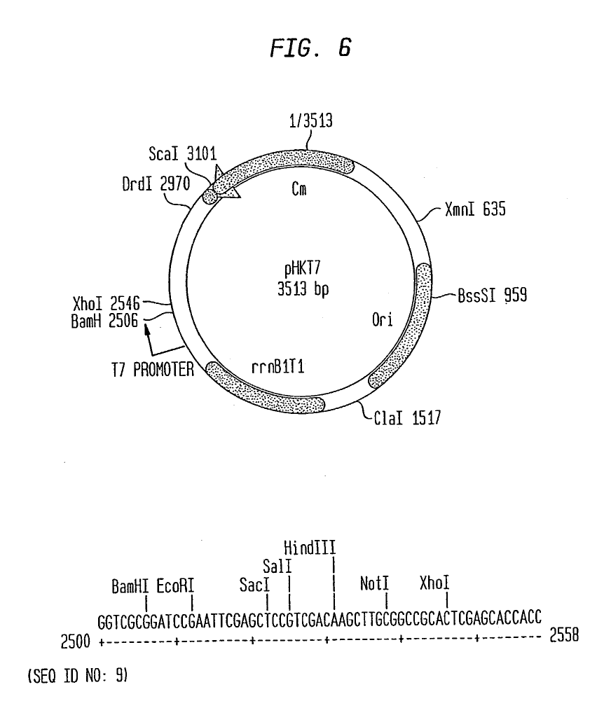

The endonuclease gene, n.bstNBIR, was cloned into pHKT7 (FIG. 6), and then introduced into E. coli ER2566 containing pHKUV5-pleIM. The culture was grown to middle log and then induced by the addition of IPTG to a final concentration of 0.4 mM. The yield of recombinant N.BstNBI endonuclease is 4×10⁷ units per gram cells.

In other embodiments, appropriate cleavage specificity for SDA is enabled by mutational alteration of enzymes having double-stranded cleavage activity. In a preferred embodiment, the sequence specificity is conferred by the specificity of a restriction enzyme, as in conventional SDA, but the strand specificity is engineered into it by mutation, so that a single purified enzyme recognizes a specific sequence and specifically nicks only one strand. Three distinct approaches to obtaining strand-specificity (nicking activity) have been devised and exemplified. Each enables performance of SDA in the absence of α-thio nucleotides. These approaches are described hereinbelow.

1. Identification of Suitable Target Enzymes for Engineering into Nicking Enzymes

Sequence-specific restriction endonucleases can be identified by methods well known in the art, and many approaches to cloning these have been devised, as described above. For the present invention, two subclasses of restriction endonucleases can be identified that are preferred starting materials for creation of sequence-specific nicking endonucleases. These will be referred to below as subclass A and subclass B. For one of these classes, the approach to obtaining mutants that nick specifically is divided into two subsets, to be referred to as subclass A1 and subclass A2. Isolation and characterization of mutants as described in subclass A is disclosed in detail in U.S. application Ser. No. ______ filed concurrently herewith and will be summarized here. Isolation and characterization of mutants of subclass B enzymes will be described in detail here.

Both classes of enzymes are found among those listed in REBASE (http://rebase.neb.com/rebase.charts.html “Type IIS enzymes” link; Roberts and Marcelis, Nucleic Acids Res. 29:368-269 (2001)) as Type IIS endonucleases. These can be identified among restriction endonucleases as those in which the recognition site is asymmetric.

However, specifically those enzymes belonging to subclass A are frequently referred to as ‘Type IIS’ endonucleases (Szybalski, Gene 100:13-26 (1991)). These enzymes recognize asymmetric sequences and cleave the DNA outside of, and to one side of, the recognition sequence. The examples that have been studied each comprise an N-terminal sequence-specific DNA binding moiety, joined with a C-terminal sequence-non-specific cleavage moiety by zero or more amino acids.

Enzymes belonging to subclass B are often referred to as ‘Type IIT’ endonucleases (Kessler, et al., Gene 47:1-153 (1986); Stankevicius, et al. Nucleic Acids Res. 26:1084-1091 (1998)), or alternately as ‘Type IIQ’ endonucleases (Degtyarev, et al., Nucleic Acids Res. 18:5807-5810 (1990); Degtyarev, et al., Nucleic Acids Res. 28:e56 (2000)). These enzymes also recognize asymmetric sequences but they cleave the DNA within the recognition sequence.

Methods for identifying and characterizing the recognition site of a restriction endonuclease are well-known in the art. In addition, a list of the known enzymes belonging to these, and other, groups may be obtained from REBASE at http://rebase.neb.com.

2. Creation of Nicking Mutants from Subclass A

The subclass A enzymes studied were FokI, MlyI, PleI, and AlwI. Enzymes of this subclass are thought to act symmetrically with respect to strand-cleavage. The C-terminal domains of two identical protein molecules are believed to interact transiently during DNA cleavage to form a homodimer.

Two of the enzymes disclosed in the present invention were derived from subclass A enzymes in one of two ways. In one preferred embodiment (method A1) cleavage of one of the two DNA strands was suppressed by mutating, within the endonuclease gene, the region coding for the dimerization interface that is needed for double-strand cleavage, such that only one cleavage occurs. This mutation may comprise alteration of particular residues required for dimerization individually or together.

In the other preferred embodiment (method A2), cleavage of one of the two strands was suppressed by substitution of the region of the endonuclease containing the dimerization interface with a corresponding region from an endonuclease known to be dimerization-defective. This region may be obtained from a portion of a gene such as the gene encoding N.BstNBI, the endonuclease of the present invention described above, or may be obtained from other naturally-occurring or from engineered genes containing this dimerization function.

3. Creation of Nicking Mutants from Subclass B

The fourth and fifth nicking endonucleases disclosed in the present invention were derived from the enzyme BbvCI, a member of subclass B. Enzymes of subclass B are thought to act asymmetrically with respect to strand-cleavage. They are envisaged to be functionally heterodimeric, that is to say to comprise two different subunits, or domains, each with its own catalytic site. In the active enzyme, the two subunits, or domains, interact to achieve DNA recognition together, and to catalyze double-strand cleavage. Of four subclass B enzymes studied-AciI, BsrBI, BssSI, and BbvCI-only BbvCI comprised two different protein subunits. The other three enzymes were single proteins each of which, we presume, comprises two different domains. In principle, nicking mutants can be made from either kind of enzyme, although doing so is more straightforward using enzymes that, like BbvCI, comprise separate, rather than joined, subunits.

A. Identification of Heterodimeric Enzymes of Subclass B

Heterodimeric members of the subclass may be recognized in two ways: by analysis of endonuclease purified from the original organism or from a recombinant host containing the cloned restriction system, or by sequence analysis of the cloned restriction system. In the former case, the purified endonuclease may be characterized by electrophoresis on SDS-PAGE, which will usually reveal the presence of two protein components migrating at different positions. It may be the case that the two subunits, although distinct in sequence and the products of different genes, still migrate at the same mobility on SDS-PAGE. This situation will be recognized, cause the apparent molecular weight derived from SDS-PAGE analysis will be one-half of the apparent molecular weight derived from gel-filtration analysis. Further, the N-terminal amino acid sequence analysis of the purified endonuclease will reveal the presence of two different amino acids at each sequencing cycle, in the apparently single band. Procedures for determining these properties are well known in the art, and are disclosed for example in Current Protocols in Protein Analysis (sections 8.3, 10.1, and 11.10; Coligan, F. E., Dunn, B. M., Ploegh, H. L., Speicher, D. W., and Wingfield, P. T. Current Protocols in Protein Science, John Wiley and Sons, (1997)).

In the latter analysis, the restriction systems amenable to this invention will contain up to four open reading frames, two encoding methyltransferases (one for each strand of the asymmetric site), and two encoding the subunits of the restriction endonuclease. The open reading frames encoding the methyltransferases may be recognized by sequence analysis according to Malone, et al., J. Mol. Biol. 253:618-632 (1995)). Additional open reading frames may also be present including those involved in the regulation of gene expression (such as C proteins), and in the repair of damage resulting from the deamination of methylated cytosine (such as Vsr proteins).

B. Verification of the Heterodimeric Character of Enzymes Identified by Sequence Analysis

Genes encoding subunits of the endonuclease may be verified by creating expression clones in which the methyltransferase genes are carried on one plasmid, and the candidate endonuclease genes are carried on one or more additional plasmid(s), as disclosed in Brooks, et al. (U.S. Pat. No. 5,320,957). Expression hosts carrying only the methyltransferase plasmid(s) will cause DNA within the cell to be resistant to action of the endonuclease, but will express no endonuclease activity. Addition of the endonuclease genes on the additional plasmid(s) will result in expression of the endonuclease activity in crude extracts of the recombinant host. In some situations it may be possible to express the endonuclease genes in the absence of the methyltransferase genes, as disclosed in WO 99/11821.

The requirement for both open reading frames for endonuclease activity may be verified by (i) creation of expression clones in which each of the two open reading frames can be expressed separately, e.g. by placing each open reading frame on a separate compatible plasmid, or by placing each open reading frame under the control of a promoter that can be induced separately (e.g. inducible by lactose or by arabinose) and then testing for expression of the endonuclease when only one open reading frame is present or only one open reading frame is expressed. Endonuclease activity will be obtained only when both open reading frames are expressed. It may also be possible to reconstitute activity by mixing extracts from two recombinant hosts expressing each open reading frame separately. The requirement for both open reading frames may alternatively be verified by (ii) creation of deletion or insertion mutations in each of the candidate open reading frames separately, followed by assessment of endonuclease activity of the resulting recombinant host. For enzymes of subclass B, both wild-type open reading frames will be required for expression of the endonuclease.

C. Converting a Heterodimeric Subclass B Enzyme to a Nicking Enzyme

Once an appropriate subclass B endonuclease has been identified, nicking enzyme derivatives pertinent to the present invention are obtained by inactivating the active site for cleavage in either subunit without interfering with the proper subsequent assembly of the enzyme. Appropriate mutations in the enzyme can be created by making mutational changes in amino acids, individually or in combination, that comprise the active site, or that influence its chemistry or organization; and then assessing the nicking activity of enzyme produced by each mutant. The magnitude of this effort may be reduced by focusing on regions conserved in several different but related enzymes.

In one preferred embodiment, changes are introduced by the steps of:

1. Identifying a conserved region by alignment of several members of this class of enzymes. Conceptual translations of five genes were employed: the two subunits of BbvCI, termed BbvCI-1, BbvCI-2, and three conventional homodimeric type II endonucleases that recognize related, palindromic, sites: Bsu36I, BlpI, and DdeI. These genes exhibit limited homology in discrete, conserved, blocks. One conserved block contained the sequence EXK. This motif was judged to be the likely active site for cleavage, in which changes may be expected to abolish cleavage but still enable assembly of a conformationally native complex in which the other subunit would still be able to cleave. These were judged favorable sites for analysis.

2. Generating mutations within the favorable region by cassette mutagenesis. This process comprised the steps of:

a) designing two mutagenic primers for inverse PCR, one for each gene, bbvCI-1 and bbvCI-2. These mutagenic primers were designed such that the nucleotides encoding the EXK motive included 20% random nucleotides, and 80% the correct nucleotide at each of the nine positions. In each mutagenic primer, the region encoding the EXK motif was flanked by the unique sequence of the respective gene;

b) conducting mutagenic PCR (as disclosed in Molecular Cloning, A Laboratory Manual, Sambrook, J. and Russel D. W., Cold Spring Harbor Laboratory, pp 8.81-8.95 (2001)) employing in separate reactions i) one mutagenic primer for bbvCI-1 and a unique primer directed in the opposite direction from the mutagenic primer and immediately to its 5′ side; and ii) one mutagenic primer for bbvCI-2 and a unique primer directed in the opposite direction from the mutagenic primer and immediately to its 5′ side, such that the entire plasmid vector was amplified;

c) ligating the PCR products to form a population circular molecules;

d) transforming an appropriate host (expressing both methyltransferases) separately with the two mutagenized populations targeting bbvCI-1 and bbvCI-2 to obtain colonies on selective plates; and

e) for isolated members of each population, testing for cleavage activity in crude extracts, by the steps of

i) growing cultures of the candidate colonies;

ii) centrifuging the cultures to obtain cell pellets;

iii) resuspending the cultures in lysis buffer;

iv) lysing the resuspended cultures and clarifying them by centrifugation;

v) withdrawing aliquots of the clarified extracts to assay tubes containing substrate plasmid DNA and digestion buffer;

vi) incubating the assay tubes to allow enzyme-induced cleavage to occur; and

vii) separating the plasmid DNA products by high-resolution gel electrophoresis and assessing whether no cleavage, single-strand cleavage, or double-strand cleavage, has occurred.

Ideally, the substrate DNA is a plasmid that contains two or more well separated sites for cleavage. Under such circumstances, extracts containing inactive enzyme do not substantially alter the mobility of the various forms of the plasmid. Extracts containing wild-type enzyme abolish the supercoiled, linear and open-circular forms of the plasmid and produce two (or more) linear fragments in their place. And extracts containing nicking enzyme abolish the supercoiled plasmid form, converting it to open-circular form, without affecting the linear form.

3. Testing mutants that appear to nick by alternative procedures to confirm that they have this activity. Such procedures include, but are not limited to, sequencing through nicked sites and sequential nicking with complementary mutants, each defective in the activity of one of the two subunits.

Most preferably, candidate enzymes are tested by the first procedure, comprising the steps of:

a) incubating DNA containing at least one site for cleavage with purified or semi-purified enzyme;

b) purifying this DNA;

c) using it as a substrate for DNA sequencing across the site in both directions.

Nicking is indicated when the sequence in one direction continues across the site (i.e., the template strand is continuous) while the sequence in the other direction terminates abruptly at the site (i.e., the other strand is interrupted by a nick).

In the second procedure, extracts of mutants thought to nick different strands are mixed together and the mixture is assayed for double-strand cleavage activity. While neither enzyme alone should catalyze double-strand cleavage, the mixture should be able to do so, either as a result of double-nicking, first on one strand by one enzyme, then on the complementary strand by the other, or by reassociation of the unmutated subunit of each enzyme to produce a fully-wild-type enzyme.

In this manner mutations in BbvCI-1 and BbvCI-2 were identified that enable cleavage of one strand but not the other at BbvCI sites. These are designated BbvCI-1-37 and BbvCI-2-12. The use of these enzymes in non-modified SDA is exemplified below.

In another embodiment, appropriate cleavage specificity for SDA is enabled by the use of enzymes having double-stranded cleavage activity, but in which cleavage occurs in two sequential steps, such that a small amount of nicked intermediate is observed during the course of double-strand cleavage.

Such enzymes that accumulate a nicked intermediate can be identified by the steps of:

a) forming a double-stranded circular substrate molecule (typically a plasmid) with one or more sites for the endonuclease;

b) incubating this substrate with small amounts of the endonuclease or for short times, such that at most 20% of substrate molecules have suffered a double-strand cleavage event;

c) separating the DNA products by high-resolution gel electrophoresis; and

d) assessing whether no cleavage, single-strand cleavage, or double-strand cleavage has occurred.

If no cleavage has occurred, in a suitable electrophoresis system containing an intercalating agent such as ethidium bromide, the substrate molecule will migrate faster than a linear DNA of the same size; if single strand cleavage has occurred, the substrate molecule will migrate slightly slower than a linear DNA of the same size; if a single double strand cleavage has occurred, the substrate molecule will migrate at the same position as a linear DNA of that size.

The nicked intermediates formed by such enzymes can support SDA as exemplified in Example 6.

The following Examples are given to additionally illustrate embodiments of the present invention as it is presently preferred to practice. It will be understood that these Examples are illustrative, and that the invention is not to be considered as restricted thereto except as indicated in the appended claims.

The references cited above and below are incorporated by reference herein.

### EXAMPLE 1

Purification of the N.BstNBI Endonuclease and Determination of its Protein Sequence

1. Purification of the N.BstNBI Restriction Endonuclease from Bacillus stearothermophilus 33M to Near Homogeneity:

Bacillus stearothermophilus 33M cells were propagated at 45° C. The cells were harvested by centrifugation after 20 hours of growth and stored at −70° C. until used. 177 g of cells were thawed at 4° C. overnight and then resuspended in 530 ml of Buffer A (20 mM KPO₄, 7 mM BME, 0.1 mM EDTA, 5% glycerol, pH 6.9) supplemented with 100 mM NaCl. The cells were broken with a Manton-Gaulin homogenizer. 25 ml of protease inhibitor cocktail (P8465; Sigma, St. Louis, Mo.) was added after the first pass. The extract was centrifuged at 14,000 rpm for 10 minutes at 4° C.

All of the following procedures were performed on ice or at 4° C. The supernatant was loaded onto a 275 ml XK 50/14 fast flow Phosphocellulose column (Whatman International Ltd., Kent, England) equilibrated with Buffer A.1 (100 mM NaCl, 20 mM KPO₄, 0.1 mM EDTA, 7 mM β-mercaptoethanol and 5% glycerol, pH 6.9). The column was washed with 2× volume of Buffer A.1, followed by a 10× linear gradient from 100 mM NaCl to 1 M NaCl in Buffer A (20 mM KPO₄, 0.1 mM EDTA, 7 mM mercaptoethanol and 5% glycerol, pH 6.9). 25 ml fractions were collected. Fractions were assayed for N.BstNBI restriction activity with T7 DNA at 55° C. in 1× N.BstNBI Buffer (150 mM KCl, 10 mM Tris-HCl, 10 mM MgCl₂, 1 mM dithiothreitol, 100 μg/ml BSA, pH 8.0). The peak of restriction enzyme activity was found to elute from the column at approximately 200 mM NaCl.

The active fractions, 39-57, were pooled (475 ml) and dialyzed against 100 mM NaCl supplemented Buffer B (20 mM Tris-HCl, 0.1 mM EDTA, 7 mM -mercaptoethanol and 5% glycerol, pH 8.0). The dialyzed pool was then diluted with Buffer B to a final concentration of 50 mM NaCl. There was a cloudy precipitate that formed but this was spun out by centrifugation in a large rotor at 14,000 rpm for 30 minutes. The cleared solution was then applied to a 22 ml HR 16/10 Source™ 15Q column (Pharmacia Biotech, Piscataway, N.J.) equilibrated in Buffer B.1 (50 mM NaCl, 20 mM Tris-HCl, 0.1 mM EDTA, 7 mM β-mercaptoethanol and 5% glycerol, pH 8.0). The column was washed with 2× volume of buffer B1 followed by a 10× linear gradient from 50 mM NaCl to 800 mM NaCl in Buffer B. 10 ml fractions were collected. Fractions were assayed for N.BstNBI activity as above. The majority of the restriction enzyme activity flowed through the column. However, fractions 6-10, which eluted at approximately 110 mM NaCl, had quite a bit of activity and were pooled (50 ml) and diluted to 50 mM NaCl in Buffer B. They were later loaded onto the second Heparin column.

The Source Q flow through and wash were combined and loaded onto a 23 ml HR 16/10 Heparin TSK-guard gel 5PW (20 μm) column (TosoHaas, Montgomeryville, Pa.) that had been equilibrated with Buffer B.2 (Buffer B with 100 mM NaCl). The column was washed with 2× volume of Buffer B.2 and then a 10× linear gradient from 100 mM NaCl to 1 M NaCl in Buffer B was performed. 7 ml fractions were collected. Fractions were assayed for N.BstNBI activity as above. Activity was found in the fractions that were eluted at approximately 550 mM NaCl. Fractions 36-39 were pooled (28 ml) and diluted to 50 mM NaCl with Buffer B.

A second HR 16/10 Heparin TSK-guard gel was then run but with diluted fractions 6-10 off of the Source Q. All conditions were the same as the first Heparin column with the only exception being that a 20× gradient was run instead of a 10× gradient. Activity was found in the fractions that were eluted at approximately 550 mM NaCl. Fractions 36-38 were pooled (21 ml) and diluted to 50 mM NaCl with Buffer B.

This pool was then combined with the pooled and diluted fractions off of the first Heparin column and loaded onto an 8 ml HR 10/10 Source™ 15Q column that had been equilibrated with Buffer B.1. The column was washed with 2× volume of Buffer B-1 and then a 15× linear gradient from 50 mM NaCl to 800 mM NaCl in Buffer B was performed. Three ml fractions were collected. Fractions were assayed for N.BstNBI activity as above. The majority of the activity flowed through. However, some activity was detected in the first 14 fractions. The flow through and wash were pooled and then fractions 1-14 were pooled (42 ml) separately from the flow through and wash. The 1-14 pool was diluted to 50 mM NaCl in Buffer B. The flow through and wash pool was run over a third Heparin column (same type as above). A 20× gradient was run from 50 mM to 1 M NaCl in Buffer B. Four ml fractions were collected. N.BstNBI was eluted at approximately 590 mM NaCl. Fractions 24-26 were pooled (12 ml) and diluted to 50 mM NaCl in Buffer A.

At the same time, pooled and diluted fractions 1-14 off of the HR 10/10 Sources 15Q were loaded onto a fourth Heparin column (same type as above). A 20× gradient was run from 50 mM to 1 M NaCl in Buffer B. 4 ml fractions were collected. N.BstNBI was eluted at approximately 590 mM NaCl. Fractions 24-26 were pooled (12 ml) and diluted to 50 mM NaCl in Buffer A.

The pooled and diluted fractions off of the third and fourth Heparin columns were combined and run over a fifth Heparin column (same type as above). Note that this time, the Heparin column was run in a phosphate buffer as opposed to a Tris-HCl buffer. The diluted pool was loaded onto the HR 16/10 Heparin TSK-guard gel column that had been previously equilibrated with Buffer A.2 (Buffer A plus 50 mM NaCl). The column was washed with a 2× volume of Buffer A.2 followed by a 20× linear gradient from 50 mM NaCl to 1 M NaCl in Buffer A. 3 ml fractions were collected. Fractions were assayed for N.BstNBI activity. The peak of the enzyme activity eluted at approximately 630 mm NaCl. Fractions 34 through 36 were pooled (9 ml) and diluted to 50 mM NaCl in Buffer A.

The diluted pool was loaded onto a 1 ml Resources 15S (Pharmacia Biotech, Piscataway, N.J.) prepacked column that had been previously equilibrated with Buffer A.2. The column was washed with a 2× volume of Buffer A.2 followed by a 20× linear gradient from 50 mM to 1 M NaCl in Buffer A. One ml fractions were collected. The majority of the activity was found in fractions 13-19 (7 ml) with the most activity being in fraction 15. The apparent salt for the elution was 750 mM NaCl; but, since the protein precipitated on the column, this isn't the “real” elution salt concentration.

The N.BstNBI was purified to approximately 80% homogeneity. Twenty μL of the peak fractions (13-18) were loaded onto an SDS-PAGE protein gel and subjected to electrophoresis. The gel was stained with Coomassie blue R-250 and a prominent band at approximately 72 kDa corresponding to the N.BstNBI restriction endonuclease activity was observed.

2. Determination of the N-terminal and Internal Protein Sequence of N.BstNBI Endonuclease

The N.BstNBI restriction endonuclease, prepared as described, was subjected to electrophoresis and electroblotted according to the procedure of Matsudaira (Matsudaira, J. Biol. Chem. 262:10035-10038 (1987)), with modifications as previously described (Looney et al., Gene 80:193-208 (1989)). The membrane was stained with Coomassie blue R-250 and the protein bands of approximately 72 kDa and 6 kDa were excised and subjected to sequential degradation on an Applied BioSystems Division, Perkin-Elmer Corporation (Foster City, Calif.) Model 407A gas phase protein sequencer (Waite-Rees et al., J. Bacteriol. 173:5207-5219 (1991)). The first 31 residues of the 72 kDa protein band corresponded to M-A-K-K-V-N-W-Y-V-S-C-S-P-W-S-P-E-K-I-Q-P-E-L-K-V-L-A-N-F-E-G (SEQ ID NO: 10) and the amino acid sequence from the N-termini of the 6 kDa internal piece of the protein was M-X-I-P-Y-E-D-F-A-D-L G (SEQ ID NO: 11).

### EXAMPLE 2

Cloning of the N.BstNBI Restriction-Modification Genes

1. Purification of Genomic DNA from Bacillus stearothermophilus 33M

To prepare the genomic DNA of Bacillus stearothermophilus 33M, 6.7 g of cells were resuspended in 20 ml of 25% Sucrose, 50 mM Tris, pH 8.0 and mixed until the solution was homogenous. Ten ml of 0.25M EDTA (pH 8.0) plus 6 ml of freshly-prepared 10 mg/ml lysozyme in 0.25M Tris-HCl (pH 8.0) were added and the solution was incubated on ice for 2 hours. Twenty four ml of Lytic mix (1% Triton-X100, 50 mM Tris, 62 mM EDTA, pH 8.0) and 5 ml of 10% SDS were then added and the solution was gently mixed. The solution was extracted with one volume of equilibrated phenol/chloroform (50:50, v/v) and the aqueous phase was recovered. The aqueous solution was then dialyzed overnight at 4° C., against 4 L of 10 mM Tris-HCl (pH 8.0), 1 mM EDTA. The dialyzed solution was digested with RNase A (100 μg/ml) at 37° C. for 1 hour. The DNA was precipitated by the addition of 1/10th volume 5 M NaCl and 0.55 volume of 2-propanol and spooled on a glass rod. The remaining solution was spun at 12,000 RPM for 30 minutes and the supernatant was then discarded. Both the spooled DNA and the centrifuged DNA pellet were air dried and dissolved in a total of 3.5 ml TE (10 mM Tris, 1 mM EDTA, pH 8.0). The final concentration was approximately 100 μg/ml and the DNA was stored at 4° C.

2. Cloning the 5′ Region of the N.BstNBI Endonuclease Gene into pCAB16

pCAB16 was digested with BsaAI by incubating the vector for 1 hour at 37° C. in the conditions described below.

120 μl PCAB 16 (6-12 μg)

10 μl BsaAI (50U)

40 μl 10× NEB Buffer #3

230 μl dH₂O

The BsaAI in the reaction was heat killed by incubating for 15 minutes at 75° C. The vector was then dephosphorylated by incubating 100 μl (2 μg) of digested vector with 1 unit of shrimp alkaline phosphatase in 100 mM MgCl₂ for 1 hour at 37° C.

Degenerate primers were designed based on the following amino acid sequences derived from the N.BstNBI N-terminal protein sequence and internal protein sequence respectively: 1) M-A-K-K-V-N-W-Y (SEQ ID NO: 12) and 2) Y-E-D-F-A-D (SEQ ID NO: 13). They were designed to hybridize in a convergent manner with DNA at the 5′ end of the N.BstNBI endonuclease gene.

Primer 1 5′ TGGCNAARAARGTNAAYTGGTA 3′ (SEQ ID NO: 14)

Primer 2 5′ TCNGCRAARTCYTCRTA 3′ (SEQ ID NO: 15)

These primers were synthesized and each was kinased by incubating 2 μg of primer with 20 units of T4 Polynucleotide Kinase, 4 μl 10× T4 Polynucleotide Kinase Buffer, and 4 μl of 10 mM ATP, in a 40 μl reaction volume at 37° C. for 30 minutes. The kinase was heat inactivated by incubating the reaction at 65° C. for 10 min.

In the reaction that was successful in amplifying the product, a reaction mix was made by combining:

10 μl of 10× NEB ThermoPol Buffer

10 μl of 2 mM dNTP solution

1.5 μl of kinased primer 1 (75 ng)

1.5 μl of kinased primer 2 (75 ng)

1 μl of purified bacterial DNA template (100 ng)

72 μl dH₂O

2 μl (4 units) of Vent®(exo−) DNA Polymerase

The PCR amplification conditions were: 32 cycles of 95° C. for 30 seconds, 45° C. for 1 minute and 72° C. for 1 minute. The reaction was electrophoresed on a 1% low melting temperature agarose gel (NuSieve Agarose, FMC BioProducts, Rockland, Me.) in TAE buffer (40 mM Tris-Acetate, pH 8, 1 mM EDTA). An approximately 1.4 Kb DNA band was excised and the gel slice was frozen overnight. The agarose plug was digested with β-Agarase by the addition of 2 μl of β-Agarase (2 units) and an incubation of 40° C. for one hour. The reaction was frozen and then thawed and microcentrifuged briefly to remove any undigested agarose pieces. The remaining aqueous layer was ethanol precipitated and the final purified DNA pellet was resuspended to 5 ng/μl. A ligation was then performed by combining the following at 37° C.:

1 μl prepared pCAB16 (50 ng)

20.5 μl PCR product (100 ng)

2.5 μl 10× T4 DNA Ligase Buffer

1 μl concentrated T4 DNA Ligase (2000 units)

The reaction was incubated at 37° C. for one hour and then it was placed in the refrigerator in an ice bucket filled with water and ice. The reaction was incubated as such overnight. Ten μl of the overnight ligation reaction was transformed into 100 μl of competent ER2502 cells by combining the DNA and cells and incubating on ice for 10 minutes followed by 45 seconds at 42° C. The entire volume was plated on an Ampicillin LB plate and incubated overnight at 37° C. Colonies that grew were inspected for the correct plasmid construct by purifying the plasmid DNA using the Qiagen QIAprep Spin Plasmid Kit and digesting with AseI to see if the PCR product was cloned into the vector.

4 μl miniprep

1.5 μl 10× NEB #3

0.5 μl AseI

9 μl dH₂O

The above reaction was incubated at 37° C. for one hour. Minipreps containing the correct size insert were sequenced. The DNA sequence was translated in six reading frames to check whether the deduced amino acid sequence corresponded with the N-terminal sequence of N.BstNBI protein.

3. Chromosome Walking via Inverse PCR to Isolate the N.BstNBI Endonuclease and Methylase Genes

A. Genomic DNA preparation. Two templates were prepared for two consecutive inverse PCR reactions; HincII and SspI. In the case of HincII, 1.5 μg of bacterial DNA was digested with 50 units of HincII restriction endonuclease in 1× NEBuffer 3 supplemented with BSA to a final concentration of 0.1 mg/ml in a 50 μl reaction volume. In the case of SspI, 1.5 μg of bacterial DNA was digested with 25 units of SspI restriction endonuclease in 1× NEBuffer SspI in a 50 μl reaction volume. Both reactions were incubated at optimum temperatures for one hour. The digests were confirmed by running 13 μl of the digestion reaction on a 1% agarose gel. The remaining reactions were then heat killed by incubating at 65° C. for 20 minutes. Circularization was then achieved by incubating the remaining 37 μl (˜1 μg) in 1× T4 DNA Ligase Buffer with 3000 units of T4 DNA Ligase in a 500 μl reaction volume at 16° C. overnight. A portion of this circularization ligation reaction was then used as the template for subsequent inverse PCR reactions.

B. HincII inverse PCR—Inverse PCR primers were synthesized based on the DNA sequence of the piece of N.BstNBI endonuclease gene cloned into pCAB16:

| 5′-CTCTTCATCAATAACGAAGTTGTT-3′ | (SEQ ID NO:16) |
|---|---|
| (221-85) | |
| 5′-TTACAACCAGTTACTCATGCCGCAG-3′ | (SEQ ID NO:17) |
| (221-86) | |

Inverse PCR was carried out using primers 221-85 and 221-86 and the above mentioned HincII DNA template. An approximately 650 base pair product was produced. This product was gel purified and resuspended in 30 μl dH₂O. The PCR product was then sequenced using an ABI 373 automated sequencing system according to the manufacturer's instructions. The PCR primers above were used as the sequencing primers. The HincII inverse PCR product contained approximately 410 novel bp of the N.BstNBI ORF.

C. SspI inverse PCR reaction—Two inverse PCR primers complementary to sequence read from the HincII inverse PCR product were synthesized (see below) and a second inverse PCR reaction was performed. Template preparation, inverse PCR, purification and DNA sequencing were all done the same as above with the exception that the SspI ligation was used to create the template as opposed to the HincII ligation. An approximately 2.2 Kb PCR product was generated and sequenced. The data revealed the remaining endonuclease ORF sequence and the n.bstNBIM DNA sequence.

| 5′ GAGTGTGAAAGAAAATATACTCAA 3′ | (SEQ ID NO:18) |
|---|---|
| (222-145) | |
| 5′ TATAGTTGTTCGATATAATGAGACCAT 3′ | (SEQ ID NO:19) |
| (222-146) | |

### EXAMPLE 3

Expression of the N.BstNBI Restriction Endonuclease

1. Cloning the PleI Methylase on a Compatible Vector

The PleI methylase gene (pleIM) was expressed by inserting the gene into an expression vector, pHKUV5, directly downstream of the strong UV5 promoter (FIG. 5). To accomplish this, two oligonucleotide primers were synthesized utilizing the DNA sequence data. The forward oligonucleotide primer contained a PstI site to facilitate cloning, a stop codon in frame with the lacZ gene to terminate translation of the lacZ protein, a ribosome binding site (RBS) and 25 nucleotides complementary to Pseudomonas lemoignei DNA for hybridization:

| 5′-AAAACTGCAGATAAGGAGGTGATCGTATGAAGCCATTAGTTAAATATAGAG-3′ | (SEQ ID NO:20) |
|---|---|
| (212-180) | |

The reverse primer was designed to hybridize to Pseudomonas lemoignei DNA at the 3′ end of the PleI gene. It contained a BamHI restriction site to facilitate cloning.

(SEQ ID NO:21)

5′-CGCGGATCCTCAATAATTTGCAACAACTATATG-3′
(212-175)

These two primers were used to amplify the pleIM gene from genomic Pseudomonas lemoignei DNA by combining:

10 μl 10× Vent® ThermoPol Buffer

10 μl of 2 mM dNTPs

4 μl (300 ng) Pseudomonas lemoignei genomic DNA

1 μl primer 212-180 (75 ng)

1 μl primer 212-175 (75 ng)

72 μl dH₂O

1 μl (0.1 units) Deep Vent® polymerase

1 μl Taq DNA polymerase (5 units)

and amplifying for 25 cycles at 94° C. for 5 minutes, 50° C. for 1 minute and 72° C. for 2 minutes. The amplification product was purified using the Promega Wizard PCR Prep Kit (Madison, Wis.). 500 ng of pHKUV5 vector and the remaining PCR product (˜2 μg) were both digested with 20 units of BamHI and 20 units of PstI, supplemented with 0.1 mg/ml BSA in 1× NEB BamHI buffer in a 60 μl reaction that was incubated at 37° C. for one hour. The digests were run on a 1% low melting temperature NuSieve agarose gel in TAE buffer. The PCR and vector DNA bands were cut out of the gel. The plasmid gel slice was treated with β-Agarase for one hour at 40° C. It was then frozen and thawed and the remaining solid gel pieces were quickly spun out using a microcentrifuge. The supernatant was ethanol precipitated and the final DNA pellet was resuspended in water. The DNA concentration was determined by visual inspection on an agarose gel. The methylase PCR was not gel purified as the vector was. The gel plug containing the methylase PCR product was used directly in the ligation reaction. The ligation of pHKUV5 and pleIM was accomplished by combining the following:

5 μl prepared pHKUV5 (100 ng)

5 μl methylase PCR product (100 ng)

1 μl Beta-Agarase (1 unit)

5 μl 10× T4 DNA Ligase Buffer

1 μl concentrated T4 DNA Ligase (2000 units)

33 μl dH₂O

The reaction was incubated at 37° C. for one hour and ten μl of the ligation reaction was transformed into E. coli strain ER2502. Individual colonies were isolated and analyzed by digesting minipreps with the cloning enzymes to ensure that the methylase gene had indeed been cloned into the vector:

3 μl miniprep

1.5 Iμl 10× BamHI buffer

1.5 μl 1 mg/ml BSA

0.75 μl PstI (15 U)

0.75 μl BamHI (15 U)

7.5 μl dH₂O

The digests were incubated at 37° C. for one hour.

The minipreps that were the correct construct were then digested with PleI to check for methylase protection:

3 μl miniprep

1.5 μl 10× NEBuffer 1

1.5 μl 1 mg/ml BSA

1 μl PleI (1 unit)

8 μl dH₂O

The digests were incubated at 37° C. for one hour. One μl of a clone that was resistant to PleI digestion was transformed into ER2566 cells for the purpose of making calcium chloride competent cells.

2. Cloning and Expression of the N.BstNBI Endonuclease Gene

The N.BstNBI endonuclease gene (n.bstNBIR) was expressed by inserting the gene into an expression vector, pHKT7, directly downstream of a strong inducible T7 promoter and a conserved ribosome binding site (RBS). To accomplish this, two oligonucleotide primers were synthesized utilizing the DNA sequence data. The forward oligonucleotide primer contained a BamHI site to facilitate cloning, an ATG start codon of the N.BstNBI endonuclease gene and 24 nucleotides complementary to Bacillus stearothermophilus 33M DNA for hybridization:

| 5′-CGCGGATCCTAAGGAGGTGATCTAATGGCTAAAAAAGTTAATTGGTAT-3′ | (SEQ ID NO:22) |
|---|---|
| (223-138) | |

The reverse primer was designed to hybridize to Bacillus stearothermophilus 33M DNA at the 3′ end of the n.bstNBIM gene. It contained a HindIII restriction site to facilitate cloning.

(SEQ ID NO:23)

5′-CCCAAGCTTTTAAAACCTTACCTCCTTGTCAAC-3′
(223-139)

These two primers were used to amplify the n.bstNBIM gene from Bacillus stearothermophilus 33M genomic DNA by combining:

15 μl 10× Taq PCR Buffer (containing 1.5 mM Mg++)

15 μl 2 mM dNTPs

3 μl (240 ng) Bacillus stearothermophilus 33M genomic DNA

1.5 μl primer 223-138 (112.5 ng)

1.5 μl primer 223-139 (112.5 ng)

111 μl dH₂O

1.5 μl (0.075 units) Deep Vent® polymerase

1.5 μl Taq DNA polymerase (7.5 units)

and amplifying for 25 cycles at 94° C. for 30 seconds, 50° C. for 1 minute and 72° C. for 2 minutes. The amplification product was purified using the Qiagen PCR Purification Kit. 1 μg of pHKT7 vector and the remaining PCR product (˜2 μg) were both digested with 20 units of BamHI and 20 units of HindIII, supplemented with 0.1 mg/ml BSA in 1× NEB Ba buffer. The reactions were incubated at 37° C. for one hour. The digests were run on a 1% low melting-point NuSieve agarose gel in TAE buffer. The PCR and vector DNA bands (approximately 1.8 Kb and 3.5 Kb respectively) were cut out and the gel slices were incubated at 65° C. for 10 minutes. The temperature was reduced to 37° C. and the gel slices were ligated. The ligation of pHKT7 and n.bstNBIM was performed by combining the following at 37° C.:

5 μl pHKT7 gel slice (50 ng)

5 μl endonuclease PCR product gel slice (100 ng)

2.5 μl 10× T4 DNA Ligase Buffer

1.5 μl T4 DNA Ligase (600 units)

1 μl Beta-Agarase (1 unit)

10 μl dH₂O

The reaction was incubated at 37° C. for one hour and then at 25° C. for another hour. Ten μl of the ligation reaction was transformed into E. coli strain ER2566 previously modified with the PleI methylase gene. Transformants were analyzed and all contained the n.bstNBIM gene. This plasmid construct, pHKT7-n.bstNBIM, was selected for producing the N.BstNBI endonuclease. The E. coli strain which contains both pHKT7-n.bstNBIR and pHKUV5-pleIM plasmids was designated as NEB#1239. The yield of recombinant N.BstNBI from strain NEB#1239 was approximately 4×10⁷ units/gram of cells.

3. Producing the Recombinant N.BstNBI Restriction Endonuclease from E. coli ER2566 NEB#1239

E. coli ER2566 NEB#1239 was grown to mid-log phase in a fermenter containing L-broth medium with ampicillin (100 μg/ml) and chloramphenicol (50 μg/ml). The culture was induced by the addition of IPTG to a final concentration of 0.4 mM and allowed to continue growing for 16 hours. The cells were harvested by centrifugation and were stored at −70° C.

Purification of the N.BstNBI restriction endonuclease from E. coli NEB#1239 can be accomplished by a combination of standard protein purification techniques, such as affinity-chromatography or ion-exchange chromatography, as outlined in Example 1 above. The N.BstNBI restriction endonuclease obtained from this purification is substantially pure and free of non-specific endonuclease and exonuclease contamination.

A sample of the E. coli ER2566 NEB#1239 which contains both pHKUV5-pleIM and pHKT7-n.bstNBIR plasmids has been deposited under the terms and conditions of the Budapest Treaty with the American Type Culture Collection on May 26, 2000 and received ATCC Accession No. PTA-1925.

### EXAMPLE 4

Non-Modified Strand Displacement Amplification Using N.BstNBI

For strand displacement amplification (SDA) to work, a nick has to be introduced into the DNA template by a restriction enzyme.

Most restriction endonucleases make double stranded breaks and therefore, α-thio dNTPs have to be used in SDA. We have tested the nicking endonuclease N.BstNBI in non-thio SDA and we found the target DNA could be successfully amplified. The following is the detailed protocol for non-thio SDA with N.BstNBI.

1. Prepare mix A (below) in a plastic 1.5 ml tube at 4° C.:

| | | Final | | | |
|---|---|---|---|---|---|
| Reagent Stock | | Concentration | | 40 μl Volume | |
| 250 | mM KP04, (pH 7.5) | 35 | mM KPO4 | 7 | μl |
| 2 | M kCl | 100 | mM | 2.5 | μl |
| 4 | mM each dNTP mix | 200 | μM each dNTP | 2.5 | μl |
| 100 | mM DTT | 1 | mM | 0.5 | μl |
| 10 | μM Primer 40 | 0.8 | μM | 4 | μl |
| 10 | μM Primer 41 | 0.8 | μM | 4 | μl |
| 2.5 | μM bump Primer 1 | 0.05 | μM | 1 | μl |
| 2.5 | μM bump Primer 2 | 0.05 | μM | 1 | μl |
| 50 | ng/μl DNA template | 1 | ng/μl | 1 | μl |
| | H₂O | | | 16.5 | μl |

2. Denature at 100° C. for 2 minutes; incubate at 55° C. for 3 minutes to allow annealing of the primers. While these two temperature incubations are occurring, prepare mix B (below) in a separate plastic 1.5 ml tube and preincubate at 55° C. for at least 30 seconds.

| | | Final | | | |
|---|---|---|---|---|---|
| Reagent Stock | | Concentration | | 10 μl Volume | |
| 10X | NEBuffer 2 | 1X | | 5.0 | μl |
| 10 | mg/ml purified BSA | 100 | μg/ml | 0.5 | μl |
| 50 | mM MgCl₂ | 2.5 | mM MgCl2 | 2.5 | μl |
| 10 | units/μl N.BstNBI | 5 | units per 50 μl | 0.5 | μl |
| 20 | units/μl Bst DNA Pol | 10 | units per 50 μl | 0.5 | μl |
| | H₂O | | | 1 | μl |

3. Add mix A to B; continue incubation at 55° C. for 20-60 minutes, removing 10-20 μl volumes at different time points if desired; add to stop dye containing 0.2% SDS (final concentration).

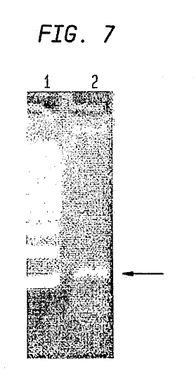

4. Analyze by gel electrophoresis on high percentage agarose gels. Specific positive bands were observed on the agarose gel (FIG. 7, Lane 1=Molecular weight standard; Lane 2=160 bp band).

5. Description of primers (all flank the polylinker region of pUC19).

| | | |
|---|---|---|
| Primer 40: | 5′-ACCGCATCGAATGCGAGTCGAGGACGACGGCCAGTG-3′ | (SEQ ID NO:24) |
| Primer 41: | 5′-CGATTCCGCAATGCGAGTCGAGGCCATGATTACGCCAA-3′ | (SEQ ID NO:25) |
| Bump primer #1: | 5′-CAGTCACGACGTT-3′ | (SEQ ID NO:26) |
| Bump primer #2: | 5′-CACAGGAAACAGC-3′ | (SEQ ID NO:27) |

6. Description of DNA template:

The templates were constructed by cloning a short DNA duplex containing SphI site into pUC19 at EcoRI and HindIII sites to generate plasmid pUC19-SphI. Lambda DNA was digested by NlaIII and ligated into plasmid pUC19-SphI pre-digested with SphI. The DNA template, which was used to produce 160-bp DNA in SDA, was screened by PCR.

### EXAMPLE 5

SDA Amplification with 5 Nicking Enzymes:

N.BstNBI, N.MlyI, N.AlwI, BbvCI #2-12 and #1-35

For strand displacement amplification (SDA) to work, a nick has to be introduced into the DNA template by a restriction enzyme.

Most restriction endonucleases make double stranded breaks and therefore, α-thio dNTPs have to be used in SDA. We have tested the nicking endonuclease N.BstNBI in non-modified SDA and we found the target DNA could be successfully amplified. The following is the detailed protocol for non-modified SDA with N.BstNBI. For N.MlyI, N.AlwI, BbvCI #2-12 and #1-35 non-modified SDA, modifications were made in the protocol in terms of the amount of enzyme used, the KCl and Mg concentrations, the assay temperature, the forward and reverse primers and the enzyme used to precut the plasmid template DNA. These modifications from the basic N.BstNBI non-modified SDA protocol are listed in part 4 of this Example.

Non-Modified SDA Protocol for N.BstNBI (with Modifications for Other Enzymes Listed)

1. Prepare mix A (below) in a plastic 1.5 ml tube at 4° C.:

| | | Final | | 35 ul | |
|---|---|---|---|---|---|
| Reagent Stock | | Concentration | | Volume | |
| 250 | mM tris, (pH 7.5) | 35 | mM tris, (pH 7.5) | 7 | ul |
| | H20 | up to volume | | 10.5 | ul |
| 2 | M KCl | 100 | mM | 2.5 | ul |
| 4 | mM each dNTP mix | 400 | uM each dNTP | 5 | ul |
| 10 | mM DTT | 1 | mM | 5 | ul |
| 10 | uM fw primer 33 | 0.2 | uM | 1 | ul |
| 10 | uM rv primer 34 | 0.2 | uM | 1 | ul |
| 2.5 | uM fw bump primer | 0.05 | uM | 1 | ul |
| 2.5 | uM rv bump brimer | 0.05 | uM | 1 | ul |
| 50 | ng/ul pre-cut pUCAH26* | 50 | ng per | 1 | ul |
| | | 50 | ul reaction | | |

2. Denature 100° C. 2 minutes; incubate at 53° C. for 3 minutes to allow annealing of the primers. While these two temperature incubations are occurring, prepare mix B (below) in a separate plastic 1.5 ml tube and preincubate at 55° C. for 30 seconds.

| | | Final | | |
|---|---|---|---|---|
| Reagent Stock | | Concentration | | 15 ul |
| | H20 | up to volume | | 3.5 ul |
| 1X | NEBuffer 2 | 5 | ul per | 5.0 ul |
| | | 50 | ul rxn vol | |
| 10 | mg/ml purified BSA | 100 | ug/ml | 0.5 ul |
| 100 | mM MgCl₂ | 10 | mM MgCl2 | 5.0 ul |
| 10 | units/ul N.BstNB I | 5 | units per | 0.5 ul |
| | | 50 | ul reaction | |
| 20 | units/ul Bst DNA Pol | 10 | units per | 0.5 ul |
| | | 50 | ul reaction | |

3. Add mix A to B; continue incubation at 53° C. for 25 min. Add stop dye containing 0.2% SDS (final concentration) to 20 ul of the reaction volume.

4. Modifications in this protocol for other nicking enzymes; volumes of added water adjusted accordingly.

| Assay | | | | | | | BbvCI | | | |
|---|---|---|---|---|---|---|---|---|---|---|
| Component | N.BstNBI | | N.AlwI | | N.MlyI | | #1-35 | | #2-12 | |
| Amount of enzyme | 5 | | 10 | | 10 | | 10 | | 5 | |
| units | | | | | | | | | | |
| KCl concentration | 100 | mM | 0 | mM | 50 | mM | 50 | mM | 50 | mM |
| MgCl2 | 10 | mM | 10 | mM | 5 | mM | 10 | mM | 5 | mM |
| concentration | | | | | | | | | | |
| Temperature of | 53° | C. | 53° | C. | 53° | C. | 45° | C. | 45° | C. |
| assay | | | | | | | | | | |

| | | | | | |
|---|---|---|---|---|---|
| Fw and Rv primer | P33, 34 | P47, 48 | P33, 34 | P49, 50 | P51, 52 |
| sets | | | | | |
| Pre-cut plasmid | Precut | Precut | Precut | Precut | Precut |
| templates | by PleI | by AlwI | by PleI | by | by |
| (eliminates | | | | PleI* | PleI* |
| endogenous nick | | | | | |
| sites) | | | | | |

*no endogenous BbvCI sites in pUC19; precutting not necessary

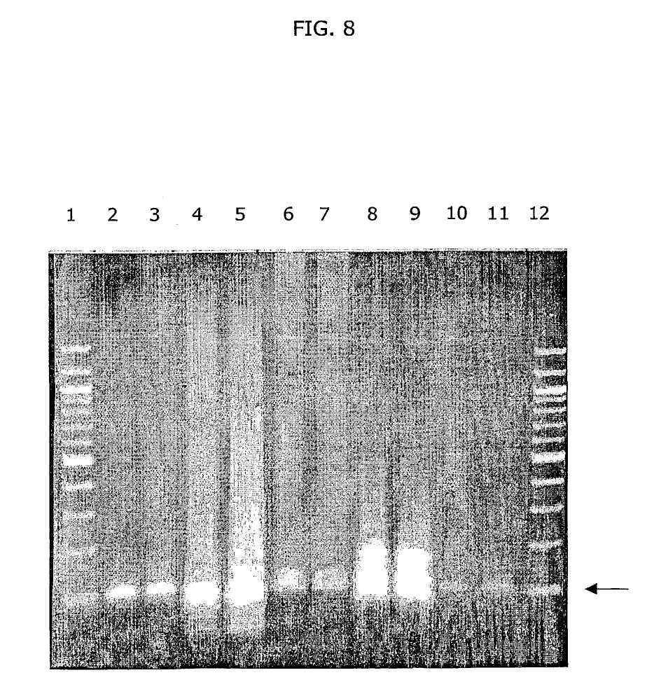

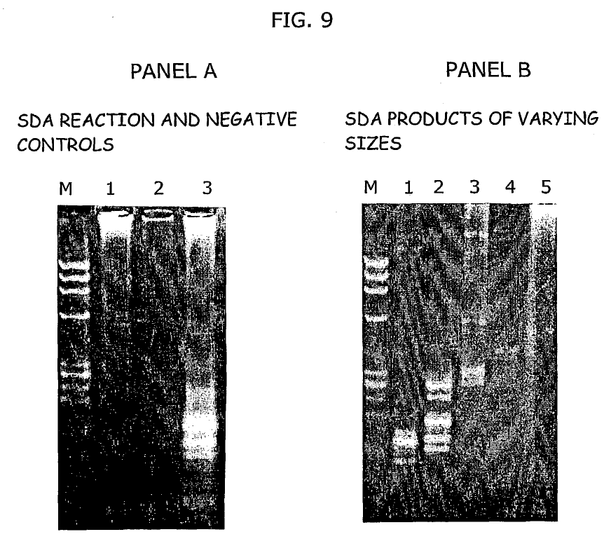

5. Analyze by gel electrophoresis on 1.5-1.8% agarose, or polyacrylamide gels. Specific 130-110 bp products were observed on the 1.8% agarose gel. (FIG. 8).

6. Description of primers (all flank the polylinker region of pUC19).

Bump Primers Used with All 5 Nicking Enzymes:

Bump Forward Primer:

5′-CAGTCACGACGTT-3′ (SEQ ID NO: 26)

Bump Reverse Primer:

5′-CACAGGAAACAGC-3′ (SEQ ID NO: 27)

Primers Specific to the Nicking Enzymes:

N.BstNB I and N.Mly I Primers:

P33Forward:

5 ′-ACCGCATCGAATGCGAGTCATGTTACGACGGCCAGTG-3′ (SEQ ID NO: 28)

P34Reverse:

5′-CGATTCCGCTCCAGGAGTCACTTTCCATGATTACGCCAA-3′ (SEQ ID NO: 29)

N.Alw I Primers:

P47Forward:

5′-ACCGCATCGAATGCGGATCATGTTACGACGGCCAGTG-3′ (SEQ ID NO: 30)

P48Reverse:

5′-CGATTCCGCTCCAGGGATCACTTTCCATGATTACGCCAA-3′ (SEQ ID NO: 31)

BbvC I, #1-35 Primers:

P49Forward:

5′-ACCGCATCGAATATGTATCGCCCTCAGCTACGACGGCCAGTG-3′ (SEQ ID NO: 32)

P50Reverse:

5′-CGATTCCGCTCCAGACTTATCCCTCAGCTCCATGATTACGCCAA-3′ (SEQ ID NO: 33)

BbvCI, #2-12 Primers:

P51Forward:

5′-ACCGCATCGAATATGTATCGCGCTGAGGTACGACGGCCAGTG-3′ (SEQ ID NO: 34)

P52Reverse:

5′-CGATTCCGCTCCAGACTTATCGCTGAGGTCCATGATTACGCCAA-3 (SEQ ID NO: 35)

7. Description of DNA Template:

The templates were constructed by cloning a short DNA duplex containing a SphI site into pUC19 at the EcoRI and HindIII sites to generate plasmid pUC19-SphI. λDNA was digested by NlaIII and ligated into plasmid pUC19-SphI pre-digested with SphI. After selecting for different sized inserts into the vector backbone, a family of plasmids was selected that could be used in SDA protocols to generate different product lengths. The specific template used in this example, pUCAH26, generates a product length of 130-110 bp (product lengths before or after nick in SDA).

### EXAMPLE 6

SDA Amplification with a Restriction Endonuclease Possessing a Strong Nicking Intermediate, such as BsrFI

For strand displacement amplification (SDA) to work, a nick has to be introduced into the DNA template by a restriction enzyme. Most restriction endonucleases make double stranded breaks and therefore, modified nucleotides such as α-thio dNTPs have to be used in SDA. We have tested the nicking endonuclease N.BstNBI in non-modified SDA and we found the target DNA could be successfully amplified (Example 4). Another approach utilizes a restriction endonuclease possessing a strong nicking intermediate. Such enzymes, when provided with a supercoiled plasmid substrate, show an accumulation of a nicked circular DNA intermediate (one strand cut) before linearization of the DNA substrate (both strands cut). We tested a variety of thermostable restriction endonucleases for their ability to produce a nicking intermediate from a supercoiled plasmid substrate as a function of time, and developed an SDA protocol using one of these enzymes, BsrFI. The BsrFI restriction endonuclease accumulates a ten-fold higher level of nicked intermediate DNA products to linearized products as a function of time.

Non-thio SDA Protocol Utilizing a Restriction Enzyme Possessing a Strong Nicking Intermediate, BsrFI

1. Prepare mix A in a plastic Eppendorf tube:

| | | Final | | | |
|---|---|---|---|---|---|
| Reagent Stock | | Concentration | | 35 ul Volume | |
| 250 | mM KP0₄, (pH 7) | 35 | mM KPO4 (pH 7) | 7 | ul |
| | H20 | up to volume | | 18-13 | ul |
| 500 | mM KCl | 0-50 | mM | 0-5 | ul |
| 4 | mM each dNTP mix | 400 | uM each dNTP | 5 | ul |
| 10 | uM forward primer | 0.2 | uM | 1 | ul |
| 10 | uM reverse primer | 0.2 | uM | 1 | ul |
| 2.5 | uM bump primer | 0.05 | uM | 1 | ul |
| 2.5 | uM bump primer | 0.05 | uM | 1 | ul |
| 50 | ng/ul BsrFI precut | 50 | ng per | 1 | ul |

| | | |
|---|---|---|
| DNA plasmid template | 50 | ul reaction |

2. Denature 100° C. 2 minutes; incubate at 55° C. for 3 minutes to allow annealing of the primers. While these two temperature incubations are occurring, prepare mix B (below) in a separate plastic 1.5 ml tube and preincubate at 55° C. for 30 seconds.

| Reagent Stock | | Final Concentration | | 15 ul |
|---|---|---|---|---|
| | H20 | up to volume | | 5.5 ul |
| 1X | NEBuffer 2 | 5 | ul per | 5.0 ul |
| | | 50 | ul rxn vol | |
| 10 | mg/ml purified | 100 | ug/ml | 0.5 ul |
| | BSA | | | |
| 50 | mM MgCl₂ | 2.5 | mM MgCl2 | 2.5 ul |
| 20 | units/ul BsrF I | 10 | units per | 0.5 ul |
| | | 50 | ul reaction | |
| 10 | units/ul Bsl DNA | 10 | units per | 1.0 ul |
| | Pol | 50 | ul reaction | |

3. Add mix A to B; continue incubation at 55° C. for 20-60 min. Add stop dye containing 0.2% SDS (final concentration) to 20 ul of the reaction volume to stop the reaction.

4. Analyze by gel electrophoresis on 1.5-1.8% agarose, or polyacrylamide gels. Specific 140-500 bp products were observed on the 1.8% agarose gel. (See section 7.)

5. Description of primers (all flank the polylinker region of pUC19).

Bump Primers:

| | |
|---|---|
| Bump forward primer: | |
| 5′-CAGTCACGACGTT-3′ | (SEQ ID NO:26) |
| Bump reverse primer: | |
| 5′-CACAGGAAACAGC-3′ | (SEQ ID NO:27) |

Primers Specific to BsrFI:

P13 Forward:

5′-ACCGCATCGAATGCATGTACCGGCTACGACGGCCAGTG-3′ (SEQ ID NO: 36)

P14 Reverse:

5′-CGATTCCGCTCCAGACTTACCGGCTCCATGATTACGCCAA-3′ (SEQ ID NO: 37)

6. Description of DNA Template:

The templates were a family of pUC19-modified plasmids. The endogenous single BsoBI and BamHI sites were eliminated by cut and subsequent fill-in reactions (elimination of the BamHI site was unrelated to this project), to form pRK22. Other related constructs were made by insertion of MspI-pBR322 fragments into AccI site of the pRK22 polylinker. This generated a family of related plasmids containing different lengths of inserts in the region of DNA amplified during SDA.

What is claimed is:

1. Isolated DNA coding for the N.BstNBI restriction endonuclease, wherein the isolated DNA is obtainable from ATCC Accession No. PTA-1925.

2. Isolated DNA coding for the PleI methylase, wherein the isolated DNA is obtainable from ATCC Accession No. Pta-1925.

3. The isolated DNA of claim 2, wherein the DNA comprises SEQ ID NO: 6.

4. A vector comprising isolated DNA selected from the group consisting essentially of SEQ ID NO: 2, SEQ ID NO: 4, and SEQ ID NO: 6.

5. A host cell transformed by the vectors of claim 4.

6. A method of producing an N.BstNBI restriction endonuclease comprising culturing a host cell transformed with the vector of claim 4 under conditions suitable for expression of said endonuclease.

7. A method for strand displacement amplification in the absence of modified nucleotide comprising employing a restriction endonuclease which does not require modified nucleotides to nick double-stranded DNA on a single DNA strand.

8. Isolated DNA of claim 1, wherein the DNA comprises SEQ ID NO: 2.

9. Isolated DNA coding for the N.BstNBI DNA methylase, wherein the isolated DNA is obtainable from ATCC Accession No. PTA-1925.

10. Isolated DNA of claim 9, wherein the DNA comprises SEQ ID NO: 4.

11. A method of making a mutated Type IIT endonuclease which has nicking activity comprising the steps of:
(a) identifying a heterodimeric Type IIT endonuclease;
(b) identifying a conserved region within said Type IIT endonuclease;
(c) generating at least one mutation within said conserved region; and
(d) analyzing the mutant endonuclease of step (c) for nicking endonuclease activity.
